(19) United States
(12) Patent Application Publication
(10) Pub. No.: US 2006/0142233 A1
(43) Pub. Date: Jun. 29, 2006

(54) Gene expression and genetic changes implicated in alcoholism

(76) Inventors: Ting-Kai Li, Bethesda, MD (US); Lucinda G. Carr, Greenwood, IN (US); Julie A. Ellison, La Mesa, CA (US); Hwai Wen Chang, San Marcos, CA (US); David D. Lo, Del Mar, CA (US)

Correspondence Address: DAVID R PRESTON & ASSOCIATES APC, 12625 HIGH BLUFF DRIVE, SUITE 205, SAN DIEGO, CA, 92130, US

(21) Appl. No.: 11/269,347

(22) Filed: Nov. 8, 2005

Related U.S. Application Data
(60) Provisional application No. 60/626,362, filed on Nov. 9, 2004.

(51) Int. Cl. A61K 48/00

(52) U.S. Cl. 514/44

(57) ABSTRACT

Polynucleotides, polypeptides, kits and methods are provided related to regulated genes characteristic of alcoholism.

### RELATED APPLICATIONS

This application claims benefit of priority to U.S. Provisional Application Ser. No. 60/626,362, filed Nov. 9, 2004, entitled “Gene Expression and Genetic Changes Implicated In Alcoholism” which is incorporated herein be reference in its entirety.

This work was supported in part by U.S. Public Service Grants AA10707, AA07611, and AA00285.

### BACKGROUND OF THE INVENTION

Alcohol is a factor in a substantial number of medical problems. The consequences of alcohol abuse affect almost every part of the body. The liver, the primary source of alcohol metabolism, is severely affected by alcohol abuse. Types of alcoholic liver damage include fatty liver, alcoholic hepatitis and fibrosis and cirrhosis. It is estimated that alcoholics with alcoholic liver disease lose about 9 to 22 years of potential life. Moderate alcohol intake may decrease the risk of coronary heart disease; however, heavy drinking is linked with hypertension, weakened heart muscle, and arrhythmias. Chronic alcohol abuse depresses the immune system and results in a predisposition to infectious diseases. Alcohol use has multiple neurologic effects, including disruptive effects on cognitive and motor functioning, nutritional diseases of the nervous system, neurologic disorders consequent to alcoholic liver disease and in many cases, neurodegeneration.

Nearly all of alcohol's effects have an economic impact. Excess alcohol consumption may contribute to poor health, which in turn causes pain and suffering, generates treatment costs, increases health insurance premiums and public expenditures on health care, and causes loss of work time. Alcohol is a contributing factor in many accidents and injuries and leads to pain and suffering, costs for medical care, premature deaths, property damage, lost work time, and increased insurance premiums. There are deleterious effects in the workplace through absenteeism and lower quality of work. Alcohol may contribute to criminal activity, which in turn generates costs associated with victimization, incarceration, and increased demands on the criminal justice system. Early drinking may interfere with educational accomplishment and limit occupational achievement and earnings. Domestic relationships may be disrupted and lead to emotional distress, which affects purchasing patterns. The cost associated with alcohol abuse is incomplete, but in 1988, the cost to society was estimated to be $85.8 billion.

A number of twin and adoption studies have shown convincingly that the interaction of genetic and environmental factors determines vulnerability to alcoholism. Because of these findings, researchers are pursuing identification of the specific genes that convey risk for developing alcohol dependence. Identifying the gene or genes responsible for susceptibility to alcoholism will allow insight into the gene's actions and how they are regulated. This will help ascertain the identity of persons at high risk for alcoholism, implement intervention strategies at an early stage, and develop new treatments for alcohol-related problems.

Human genetic studies are inherently very difficult and expensive. Because of this, investigators are using genetic animal models as tools to elucidate the genes that influence alcohol-drinking behavior. There are several advantages of using genetic animal models. Compared to human research, genetic experiments with animal models are relatively quick and inexpensive. The researcher has control of the mating behavior and thus, genetically influenced biological characteristics of alcoholism observed in humans can be created in animal models. In addition, it is often possible to test alcohol-drinking behavior hypotheses in animals that are not possible in humans because of ethical reasons. The use of animal models to identify genes underlying alcohol-seeking behavior will provide candidate genes that can then be tested in human studies to determine whether they have an effect on the human alcoholism phenotype. What is needed therefore, is an understanding and identification of the genes associated with alcoholism.

### SUMMARY OF THE INVENTION

Despite advances in recent years, the precise etiology and pathogenesis of alcoholism remains undefined. In order to try to better understand the mechanistic basis of alcoholism, much effort has been directed towards the discovery and development of various animal models of alcoholism. Several rat strain lines have been selectively bred for differential ethanol drinking behaviors. These lines were generated by selectively breeding animals for a particular phenotypic trait and then, in some cases, completing many generations of brother-sister mating to produce highly inbred strains with the phenotype of interest. The alcohol-preferring (P) and alcohol-non-preferring (NP) rat lines were developed at Indiana University for high and low alcohol preference behavior through bidirectional selective breeding from a randomly bred closed colony of Wistar rats (Wrm: WRC(WI)BR) from the Walter Reed Army Institute of Research, Washington, D.C. (Li et al, 1991). Following successful divergence and plateau of the phenotype in each of the selected lines, brother-sister mating was initiated at generation 30 of selection to develop inbred lines.

The TOtal Gene expression Analysis (TOGA®) method, described in Sutcliffe et al., Proc. Natl. Acad. Sci. USA 97(5): 1976-81 (2000), WO 00/26406, U.S. application Ser. No. 09/775,217, PCT/US02/02666, U.S. Pat. No. 5,459,037, U.S. Pat. No. 5,807,680, U.S. Pat. No. 6,030,784, U.S. Pat. No. 6,309,834, U.S. Pat. No. 6,596,484, U.S. Pat. No. 6,633,818, U.S. Pat. No. 6,096,503, U.S. Pat. No. 6,110,680, and U.S. Pat. No. 6,309,834, all of which are incorporated herein by reference, is a tool used to identify and analyze polynucleotide expression associated with alcoholism. The TOGA® method is an improved method for the simultaneous sequence-specific identification of mRNAs in an mRNA population which allows the visualization of nearly every mRNA expressed by a tissue as a distinct band on a gel whose intensity corresponds roughly to the concentration of the mRNA. The method can identify changes in expression of mRNA associated with the administration of drugs or with physiological or pathological conditions, such as alcoholism.

The present invention associates previously known polynucleotides, their corresponding genes and regions thereof and their encoded polypeptides to alcoholism such that the polynucleotides, polypeptides, genes and regions thereof can be useful for diagnosis and treatment of alcoholism. Some embodiments of the invention provide methods for preventing, treating, modulating, or ameliorating a medical condition, such as alcoholism, comprising administering to a mammalian subject a therapeutically effective amount of at least one polypeptide of the invention, at least one polynucleotide of the invention, at least one gene of the invention, or a region thereof. A preferred embodiment of the invention provides a method for preventing, treating, modulating, or ameliorating a medical condition, such as alcoholism, comprising administering to a mammalian subject a therapeutically effective amount of an antibody that binds specifically to a polypeptide of the invention.

Additional embodiments of the invention provide a method for using a polynucleotide of the invention, a polypeptide of the invention, an antibody of the invention, or a gene of the invention or a region thereof for the manufacture of a medicament useful in the treatment of alcoholism. An additional embodiment of the invention provides a method of diagnosing alcoholism or susceptibility to alcoholism in a subject. The method comprises determining the presence or absence of a mutation in a polynucleotide or gene of the invention or a region thereof. Alcoholism or a susceptibility to alcoholism is diagnosed based on the presence of the mutation.

Even other embodiments of the invention provide methods of diagnosing alcoholism or a susceptibility to alcoholism in a subject. The methods comprise detecting an alteration in expression of a polynucleotide, gene or region thereof, or a polypeptide encoded by the polynucleotide or gene of the invention, wherein the presence of an alteration in expression of the polypeptide is indicative of alcoholism or susceptibility to alcoholism. The alteration in expression can be an increase in the amount of expression or a decrease in the amount of expression. In a preferred embodiment, a first biological sample is obtained from a patient suspected of having a susceptibility to alcoholism and a second sample from a suitable comparable control source is obtained. The amount of at least one polypeptide, polynucleotide or gene of the invention or a region thereof is determined in the first and second sample. A patient is diagnosed as having a susceptibility to alcoholism if the amount of the polypeptide, polynucleotide or gene or region thereof in the first sample is greater than or less than the amount of the polypeptide, polynucleotide or gene or region thereof in the second sample.

Where a polynucleotide or gene of the invention is down-regulated and is associated with alcoholism, the expression of the polynucleotide or gene can be increased or the level of the intact polypeptide product can be increased in order to treat or prevent alcoholism or ameliorate symptoms associated with alcoholism, such as, for example, fatty liver, alcoholic hepatitis, fibrosis, cirrhosis, hypertension, weakened heart muscle, and arrhythmia. This can be accomplished by, for example, administering a polynucleotide, gene, or polypeptide of the invention to the mammalian subject.

Where a polynucleotide or gene of the invention is up-regulated and is associated with alcoholism in a mammalian subject, the expression of the polynucleotide or gene can be blocked or reduced or the level of the intact polypeptide product can be reduced in order to treat or prevent alcoholism or ameliorate symptoms associated with alcoholism, such as, for example, fatty liver, alcoholic hepatitis, fibrosis, cirrhosis, hypertension, weakened heart muscle, and arrhythmia. This can be accomplished by, for example, by administering an inhibitor of the polynucleotide, gene, or polypeptide of the invention to a mammalian subject such as through the use of antisense oligonucleotides, triple helix base pairing methodology or ribozymes. Alternatively, drugs or antibodies that bind to and inactivate the polypeptide product or otherwise interfere with the activity of the polypeptide in the disease state can be used.

Yet other embodiments of the invention involve assessing the stage of alcoholism by testing for regulation of at least one polynucleotide, polypeptide, antibody or gene of the invention or a region thereof. Further embodiments of the invention involve assessing the efficacy or toxicity of a therapeutic treatment for alcoholism by testing for regulation of at least one polynucleotide, polypeptide, antibody or gene of the invention or a region thereof.

Another embodiment of the present invention provides a method of using a polynucleotide, polypeptide, antibody or gene of the invention or a region thereof for delivering to a patient in need thereof, genes, DNA vaccines, diagnostic reagents, peptides, proteins or macromolecules. Another embodiment of the invention provides a method of using a polypeptide or antibody of the invention to identify a binding partner to a polypeptide of the invention. In a preferred embodiment, a polypeptide of the invention is contacted with a binding partner and it is determined whether the binding partner affects an activity of the polypeptide.

Still another embodiment of the invention provides a substantially pure isolated DNA molecule suitable for use as a probe for genes regulated in alcoholism, chosen from the group consisting of the DNA molecules shown in SEQ ID NO:1-14, or their corresponding genes or regions thereof, or DNA molecules at least 95% similar to one of the foregoing molecules.

Even another embodiment of the invention provides a kit for detecting the presence of a polypeptide of the invention in a mammalian tissue sample. In one embodiment, the kit comprises a first antibody that immunoreacts with a mammalian protein encoded by a gene corresponding to the polynucleotide of the invention or with a polypeptide encoded by the polynucleotide in an amount sufficient for at least one assay and suitable packaging material. The kit can further comprise a second antibody that binds to the first antibody. The second antibody can be labeled with enzymes, radioisotopes, fluorescent compounds, colloidal metals, chemiluminescent compounds, phosphorescent compounds, bioluminescent compounds, or with an organic moiety, such as biotin.

Another embodiment of the invention provides a kit for detecting the presence of genes encoding a protein comprising a polynucleotide of the invention, or fragment thereof having at least 10 contiguous bases, in an amount sufficient for at least one assay, and suitable packaging material.

An additional embodiment of the invention involves a method for identifying biomolecules associated with alcoholism comprising the steps of: developing a cellular experiment specific for alcoholism, harvesting the RNA from the cells used in the experiment, obtaining a gene expression profile, and using the gene expression profile for identifying biomolecules whose expression was altered during the experiment. The biomolecules identified may be polynucleotides, polypeptides or genes.

Yet another embodiment of the invention provides a method for detecting the presence of a nucleic acid encoding a protein in a mammalian tissue sample. A polynucleotide or gene of the invention or fragment thereof having at least 10 contiguous bases is hybridized with the nucleic acid of the sample. The presence of the hybridization product is detected.

The foregoing merely summarizes certain aspects of the invention and is not intended, nor should it be construed as limiting the invention in any way.

### BRIEF DESCRIPTION OF THE DRAWINGS

These and other features, aspects, and advantages of the present invention will become better understood with reference to the following description, appended claims, and accompanying drawings where:

FIG. 1 is a graphical representation of the results of TOGA® analysis using a 5′ PCR primer with parsing bases ATGG (SEQ ID NO:24) and the universal 3′ PCR primer (SEQ ID NO:17). The graph shows the results produced by TOGA® from mRNA extracted from the cortex of the P rat brain (Panel A), the cortex of the NP rat brain (Panel B), the hypothalamus of the P rat brain (Panel C), and the hypothalamus of the NP rat brain (Panel D), the hippocampus of the P rat brain (Panel E), and the hippocampus of the NP rat brain (Panel F) where the vertical index line indicates a PCR product of about 266 base pairs (b.p.) that displays differential expression among the samples, expression being much greater in all of the NP rat brain regions compared to the P brain regions. The size in base pairs of the PCR products is shown along the horizontal axis. The vertical axis represents the fluorescence intensity of the PCR products, which is a measure of the relative expression of the corresponding mRNAs.

The data from TOGA® were normalized using the methods described in pending U.S. patent application Ser. No. 09/318,699/U.S., and PCT Application Serial No. PCT/US00/14159, both entitled Methods and System for Amplitude Normalization and Selection of Data Peaks (Dennis Grace, Jayson Durham); and U.S. Pat. No. 6,334,099, PCT Application Serial No. PCT/US00/14123, and pending U.S. patent application Ser. No. 09/940,987/U.S., Ser. No. 09/940,581/U.S., Ser. No. 09/940,746/U.S., all entitled Methods for Normalization of Experimental Data (Dennis Grace, Jayson Durham), all of which are incorporated herein by reference. The vertical line drawn through the six panels indicates the location of a molecule known as CAR1_{—}3 (SEQ ID NO:1).

FIG. 2 presents a graphical example of the results obtained when a sequenced TOGA PCR product, referred to as a DST (Digital Sequence Tag), is verified by the Extended TOGA® Method using a primer generated from a cloned product (as described below). The length of the PCR product corresponding to SEQ ID NO:1 (DST CAR1_{—}3) was cloned and a 5′ PCR primer was built from the cloned DST (SEQ ID NO:25). The product obtained from PCR with this primer (SEQ ID NO:25) and the universal 3′ PCR primer (SEQ ID NO:17) (as shown in the top panel) was compared to the length of the original PCR product that was produced in the TOGA® reaction with mRNA extracted from the NP rat cortex sample using a 5′ PCR primer with parsing bases ATGG (SEQ ID NO:26) and the universal 3′ PCR primer (SEQ ID NO:17) (as shown in the middle panel). Again, for all panels, the number of base pairs is shown on the horizontal axis, and fluorescence intensity (which corresponds to relative expression) is found on the vertical axis. In the bottom panel, the traces from the top and middle panels are overlaid. The data in the bottom panel demonstrate that the peak in the top panel, generated by using an extended primer whose sequence derives from the cloned DST, migrates at the identical size position as the original PCR product obtained through TOGA® in the middle panel, identified as DST CAR1_{—}3 (SEQ ID NO:1).

FIG. 3 compares the results from Real Time PCR validation to duplicate runs of TOGA®. The graph in FIG. 3 illustrates that, during the progressive amplification cycles of the target DST CAR1_{—}3 (SEQ ID NO:1) sequence in rat cortex, detection of the amplified sequence occurs earliest in the NP rat cortex sample reflecting the fact that a greater number of RNA molecules for this sequence were available from the start. As the chart in FIG. 3 illustrates, the DST CAR1_{—}3 (SEQ ID NO:1) is found in a much greater proportion in NP rat cortex in both TOGA® assays and Real Time PCR. The relative abundance is measured as compared to the P rat cortex sample, which has the relative abundance set to 1.00.

FIG. 4 demonstrates the neuroanatomical and cellular localization of DST CAR1_{—}3 (SEQ ID NO:1), which was identified by database searches and nucleotide sequence comparisons as rat glutathione S transferase 8-8 (rGST 8-8). To identify brain regions where rGST 8-8 was expressed, in situ hybridization was performed on brain sections from the P and NP rats. rGST 8-8 was expressed throughout the brain, with expression localized to specific brain regions and cells. An example of the distinctive patterns of expressions is shown in FIG. 4A, where rGST 8-8 was highly expressed in the choroid plexus of the dorsal 3^{rd} ventricle (D3V, FIG. 4A. 1,2), the lateral ventricle (LV, FIG. 4A. 3, 4), and the CA2 and CA3 regions of the hippocampus (FIG. 4A. 3 through 6). Lower expression levels were detected in the thalamus (FIG. 4A. 4), and cortex (FIG. 4A. 6). Comparison of rGST 8-8 expression between P (FIG. 4A. 1, 3, 5) and NP (FIG. 4A. 2, 4, 6) on the autoradiographs showed lower expression in P than NP. Quantification of rGST 8-8 expression in the CA2 and CA3 regions of the P and NP hippocampus revealed a 3-fold lower expression in P (FIG. 4B. 1 and 3) than NP (FIG. 4B. 2 and 4) (p<0.0001). At the cellular level, it is clear that rGST 8-8 is expressed in the pyramidal cells of CA2 and CA3 of P and NP rat brain (FIG. 4B. 1 through 4), the endothelial cells of the choroid plexus (FIG. 4B. 5), and the ependymal cells along the dorsal third ventricle (D3V, FIG. 4B. 5). Expression of rGST 8-8 in the ependymal cells is very evident at the base of the third ventricle (3V) of NP rats (FIG. 4B. 6).

FIG. 5 illustrates rGST 8-8 protein expression analyzed using quantitative Western blot analysis on samples from the amygdala of alcohol-preferring and alcohol-non-preferring rats. The blots were probed with both rGST 8-8 antibody and neuron specific enolase (NSE) antisera. The P rat amygdala samples P1, P2, and P3 showed 1.6 fold average lower level of rGST 8-8 expression than NP rat amygdale samples NP1, NP2, and NP3. The NSE signal was used to normalize the amount of neuronal protein loaded per lane.

FIG. 6 demonstrates single nucleotide polymorphisms (SNPs) revealed by sequence analysis of the coding and 3′ untranslated region (3′ UTR) of the rGST 8-8 mRNA in two rat strains. Sequences of cDNA synthesized from rGST 8-8 mRNA revealed a silent single nucleotide polymorphism (SNP) in the coding region at +628, relative to the translation start site (+1), and three SNPs were discovered in the 3′-UTR. The P sequence displayed differences in four positions (+628, +714, +727 and +756), while the NP sequence was identical to the published rGST 8-8 cDNA sequence (Accession number: X62660). An alignment of the P and NP sequences with another published GST sequence (XM_{—}217195) indicated that the NP rGST 8-8 sequence was also more homologous to this sequence than was the P sequence.

### DETAILED DESCRIPTION OF THE PREFERRED EMBODIMENTS

The present invention and the methods of obtaining and using the present invention will be described in detail after setting forth some preliminary definitions, which are provided to facilitate understanding of certain terms used in the present invention. Many of the techniques presented herein are described in Dracopoli et al., Current Protocols in Human Genetics, John Wiley and Sons, New York (1999), and Ausubel et al., Current Protocols in Molecular Biology, John Wiley and Sons, New York (2000), both of which are incorporated herein by reference.

Definitions

“Isolated” refers to material removed from its original environment (e.g., the natural environment if it is naturally occurring), and thus is altered “by the hand of man” from its natural state.

“Stringent hybridization conditions” refers to an overnight incubation at 42° C. in a solution comprising 50% formamide, 5×SSC (5×SSC=750 mM NaCl, 75 mM sodium citrate, 50 mM sodium phosphate pH 7.6), 5× Denhardt's solution, 10% dextran sulfate, and 20 μg/ml denatured, sheared salmon sperm DNA, followed by washing the filters in 0.1×SSC at about 65° C. Changes in the stringency of hybridization and signal detection are primarily accomplished through the manipulation of formamide concentration (lower percentages of formamide result in lowered stringency); salt conditions, or temperature. For example, lower stringency conditions include an overnight incubation at 37° C. in a solution comprising 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Note that variations in the above conditions may be accomplished through the inclusion and/or substitution of alternate blocking reagents used to suppress background in hybridization experiments. Typical blocking reagents include Denhardt's reagent, BLOTTO (5% w/v non-fat dried milk in phosphate buffered saline (“PBS”), heparin, denatured salmon sperm DNA, and other commercially available proprietary formulations. The inclusion of specific blocking reagents may require modification of the hybridization conditions described above, due to problems with compatibility. Of course, a polynucleotide which hybridizes only to polyA+ sequences (such as any 3′ terminal polyA+ tract of a cDNA shown in the sequence listing), or to a complementary stretch of T (or U) residues, would not be included in the definition of “polynucleotide,” since such a polynucleotide would hybridize to any nucleic acid molecule containing a poly (A) stretch or the complement thereof (e.g., practically any double-stranded cDNA clone).

“Conservative amino acid substitution” refers to a substitution between similar amino acids that preserves an essential chemical characteristic of the original polypeptide.

“Identity” per se has an art-recognized meaning and can be calculated using published techniques. (See, e.g., Lesk, Ed., Computational Molecular Biology, Oxford University Press, New York, (1988); Smith, Ed., Biocomputing: Informatics And Genome Projects, Academic Press, New York, (1993); Griffin and Griffin, Eds., Computer Analysis Of Sequence Data, Part 1, Humana Press, New Jersey, (1994); von Heinje, Sequence Analysis In Molecular Biology, Academic Press, (1987); and Gribskov and Devereux, Eds., Sequence Analysis Primer, M Stockton Press, New York, (1991)). While there exists a number of methods to measure identity between two polynucleotide or polypeptide sequences, the term “identity” is well known to skilled artisans (Carillo et al., SIAM J Applied Math., 48:1073 (1988)). Methods commonly employed to determine identity or similarity between two sequences include, but are not limited to, those disclosed in “Guide to Huge Computers,” Martin J. Bishop, Ed., Academic Press, San Diego, (1994) and Carillo et al., (1988), Supra.

“EST” refers to an Expressed Sequence Tag, i.e. a short sequence of a gene made from cDNA, typically in the range of 200 to 500 base pairs. Since an EST corresponds to a specific region of a gene, it can be used as a tool to help identify unknown genes and map their position in the genome.

“DST” refers to a Digital Sequence Tag, i.e., a polynucleotide derived from TOGA® methodology that is an expressed sequence tag of the 3′ end of an mRNA.

Other terms used in the fields of biotechnology and molecular and cell biology as used herein will be as generally understood by one of ordinary skill in the applicable arts.

Animal models of alcohol preference have been used to identify both chromosomal loci and candidate genes that may influence alcohol seeking behavior. The animal model used in these studies, the alcohol inbred preferring (P or iP) and alcohol inbred non-preferring (NP or iNP) rat lines, was developed at Indiana University. These rats were bred from a Wistar stock with the following selection criteria for inclusion in the alcohol preferring cohort: 1) daily consumption of greater than 5.0 grams of ethanol per kilogram of body weight and 2) a preference ratio of greater than 2:1 of ethanol to water consumed. The alcohol preferring rat strain has been demonstrated to be a good model for studying the hypotheses concerning alcohol use and abuse in humans (Li et al., 1991).

Based upon observations of human drinking behavior, three behaviors define alcohol preferring behavior. One, the rewarding features of low-to-moderate amounts of alcohol is important in the initiation and continuation of drinking. Two, the aversive effects of large amounts of alcohol do not deter heavy drinking. Three, tolerance that usually develops to the aversive effects of drinking does not take place. These three behaviors have been ascertained to be present in the alcohol preferring versus non-preferring rats.

The alcohol preferring rats have phenotypic characteristics considered necessary for an animal model of alcoholism (Cicero, 1979) including: 1) high voluntary oral consumption of ethanol leading to high blood alcohol concentrations (50-250 mg %), even in the presence of food and water; 2) consumption of ethanol for its pharmacological effects, rather than its taste, smell or caloric content, as evidenced by intragastric and intracerebral self-administration of ethanol; 3) operant responding for oral consumption of ethanol at concentrations as high as 30%; and 4) development of metabolic and behavioral tolerance as well as physical dependence when allowed chronic, free choice alcohol drinking (Gatto et al., 1987; Gatto et al., 1987; Froehlich et al., 1988; Murphy et al., 1989; Li et al, 1991).

Koob et al (1998) postulated a unifying neurobiological basis underlying the reinforcing and neuroadaptive actions of the opiates, psychostimulants, and alcohol. Several brain regions have been postulated to mediate the alcohol seeking behavior that leads to alcoholism. The mesocorticolimbic dopamine (DA) system and its interaction with the extended amygdala play key roles in regulating alcohol drinking behavior, as well as the actions of the opiates and psychostimulants. The ventral tegmental area (VTA) is the major source of DA neurons in the limbic system. Reciprocal interactions between the VTA and the nucleus accumbens, ventral pallidum, prefrontal cortex, dorsal raphe nucleus and pedunculopontine nucleus appear to be involved in regulating alcohol drinking behavior (McBride and Li 1998). Information from the amygdala and hippocampus converge at the level of the nucleus accumbens and are involved in regulating its output and altering alcohol drinking behavior. The lateral hypothalamus has also been implicated in mediating reinforcement processes (see McBride et al 1999) and may also be involved in high alcohol-seeking behavior. Within the limbic structures, neurobiological, neuropharmacological and neurophysiological studies (reviewed in McBride and Li 1998) indicate the involvement of mu- and delta-opioid, GABA-A, DA D-2 and D-3, muscarinic, NMDA, neuropeptide Y, serotonin-1B, -2 and -3, and CRF receptors in mediating alcohol drinking (reviewed in McBride and Li 1998). In addition, abnormalities in the DA and serotonin (5-HT) systems projecting to the nucleus accumbens appear to be associated with the high alcohol drinking behavior of the P and HAD lines of rats (McBride and Li 1998). Finally, preliminary studies indicate that chronic alcohol drinking by the P line of rats reduces local cerebral glucose utilization (LCGU) rates in the VTA, nucleus accumbens, medial prefrontal and frontal cortices, lateral hypothalamus, amygdala and hippocampus, suggesting that chronic alcohol exposure alters neuronal activity within these structures.

Previously, a “candidate” gene approach was undertaken to identify genes that influenced the drinking behavior of the P and NP rats; a polymorphism was not identified that could be confirmed in segregating F2 progeny of the P×NP intercross. This is a very laborious approach and is quite limited compared to the TOGA® (total gene expression analysis) technology. Because the genetic basis of alcohol-seeking behavior is still largely unknown, it is very likely that there are genetic influences that are unknown (cDNAs not cloned) or not considered important at this time. Thus, a method such as TOGA® is useful because it can identify mRNAs unique between the P and NP rat lines regardless of whether the mRNA has been discovered. In addition, the automated technology allows analyses of thousands of mRNAs. This technology complements a quantitative trait loci (QTL) study being performed in the P and NP rat lines. A QTL on chromosome 4 with a maximum lod score of 9.2 has been identified (Carr et al, 1998; Bice et al, 1998). A 1-lod support interval encompasses 12.5 cM of the chromosome and thus, there are many genes in this region. It is not feasible to sequence the entire region and congenic rat lines are being generated to narrow the region.

A recent study attempted to determine whether QTLs could be identified by applying TOGA® to screen mRNA populations for RNA species whose concentrations differed in a manner that tracked the differences in phenotype and whose genes also mapped to QTL intervals. RNA from several brain regions of P and NP rats was profiled by TOGA®; in total 19,954 mRNAs were detected (Liang et al., 2003). A total of 28 mRNAs exhibited a 2-fold or greater difference between P and NP rats. One of the mRNAs, representing the gene α-synuclein, derives from a gene with a chromosomal location within the QTL interval on chromosome 4. The sequences of the α-synuclein genes from P and NP rats revealed 2 SNPs in the 3′ untranslated region of the genes, and were used to fine-map the gene using recombination-based methods to the peak of the this major QTL on rat chromosome 4.

In the study of the present invention, TOGA® was used to identify mRNAs that are differentially expressed or uniquely expressed in the alcohol-preferring and alcohol-non-preferring rats in selected regions of the brain for the purpose of quantitative trait gene discovery. The resulting genes and genetic variants detected in the study could provide useful in disease diagnosis and the proteins encoded by differentially expressed genes that map to known alcoholism-related QTLs, as with α-synuclein described above, would serve as likely targets for therapeutic intervention, or as therapeutics themselves. These genes and associated mRNAs may be associated with alcohol consumption in the P and NP rats and should provide important insights into the genetics of human alcoholism. Four brain regions were analyzed: the nucleus accumbens (striatum), cortex, hypothalamus, and hippocampus, because of their potential importance in mediating alcohol drinking behavior and the observation that chronic alcohol exposure can alter neuronal activity within these regions.

Isolated RNA was analyzed using TOGA®. The expression pattern of thousands of genes were analyzed in the striatum, cortex, hypothalamus and hippocampus of the alcohol preferring rat and compared with the expression pattern in the striatum, cortex, hypothalamus and hippocampus of the alcohol-non-preferring rat.

The PCR-based TOGA® differential display system was used in studies to determine how alcohol seeking behavior and alcoholism is regulated or influenced by genetic susceptibility or genetic predisposition in a strain of rats that were bred to prefer alcohol (alcohol-preferring) as compared to a related strain (alcohol-non-preferring). Such studies have examined the genetic basis for alcohol seeking behavior and have examined proteins and genes that may prevent alcohol seeking behavior or alcoholism. Using TOGA®, molecules have been identified that correspond to genes that are differentially expressed in alcohol-preferring rats as compared to alcohol-non-preferring rats. Such molecules are useful in therapeutic and diagnostic applications in the treatment of alcoholism.

Using the TOGA® Technology, 7 genes were identified (set forth in Table 1) as differentially regulated in the alcohol-preferring versus alcohol-non-preferring rats and studied in detail. These genes were either differentially regulated in all four brain regions (striatum, cortex, hypothalamus, hippocampus) or differentially regulated in a single brain region (set forth in Table 5). These results support the idea that specific brain regions are involved in the genetic susceptibility to alcoholism. Furthermore, 4 of the differentially regulated DSTs are increased in the alcohol preferring rats and 3 of the differentially regulated DSTs are decreased in the alcohol preferring rats. As will be demonstrated in the example below not only are the DSTs differentially regulated at the mRNA level, but this differential regulation also correlates with alterations in accumulated protein. In addition, genetic polymorphisms (single nucleotide polymorphisms, or SNPs) occur in the gene encoding GST 8-8 that distinguish P from NP rats.

Treatment of Alcoholism

Where a polynucleotide, polypeptide or gene of the invention or region thereof is down-regulated and is associated with alcoholism, the expression of the polynucleotide or gene or region thereof can be increased or the level of the intact polypeptide product can be increased in order to treat or prevent alcoholism or ameliorate symptoms associated with alcoholism, such as, for example, fatty liver, alcoholic hepatitis, fibrosis, cirrhosis, hypertension, weakened heart muscle, and arrhythmia. This can be accomplished by, for example, administering a polynucleotide, polypeptide or gene of the invention or region thereof (or a set of polynucleotides, polypeptides, genes or regions thereof, including those of the invention) to the mammalian subject.

A polynucleotide or gene of the invention or region thereof can be administered to a mammalian subject alone or with other polynucleotides or genes by a recombinant expression vector comprising the polynucleotide or gene or region thereof. As used herein, a mammalian subject can be a human, baboon, chimpanzee, macaque, cow, horse, sheep, pig, dog, cat, rabbit, guinea pig, rat or mouse. Preferably, the recombinant vector comprises a polynucleotide shown in SEQ ID NOs:1-14 or a polynucleotide which is at least 98% identical to a nucleic acid sequence shown in SEQ ID NOs:1-14 or a gene corresponding to one of the foregoing polynucleotides or a region thereof. Also, preferably, the recombinant vector comprises a variant polynucleotide that is at least 80%, 90%, or 95% identical to a polynucleotide comprising at least one of SEQ ID NOs:1-14, a polynucleotide at least ten bases in length hybridizable to polynucleotide comprising at least one of SEQ ID NOs:1-14, a polynucleotide comprising at least one SEQ ID NOs:1-14 with sequential nucleotide deletions from either the 5′ terminus or the 3′ terminus, or a species homolog of a polynucleotide comprising at least one of SEQ ID NOs:1-14 or gene corresponding to any one of the foregoing polynucleotides of a region thereof.

The administration of a polynucleotide or gene of the invention, or region thereof or recombinant expression vector containing such polynucleotide, gene or region thereof to a mammalian subject can be used to express a polynucleotide in said subject for the treatment of, for example, alcoholism. Expression of a polynucleotide or gene in target cells would effect greater production of the encoded polypeptide. In some cases, where the encoded polypeptide is a nuclear protein, the regulation of other genes may be secondarily up- or down-regulated.

There are available to one skilled in the art multiple viral and non-viral methods suitable for introduction of a nucleic acid molecule into a target cell, as described above. In addition, a naked polynucleotide, gene or region thereof can be administered to target cells. Polynucleotides and genes of the invention or regions thereof and recombinant expression vectors of the invention can be administered as a pharmaceutical composition (including, without limitation, genes delivered by vectors such as adeno-associated virus, liposomes, PLGA, canarypox virus, adenovirus, retroviruses including IL-1 and GM-CSF antagonists). Such a composition comprises an effective amount of a polynucleotide, gene or region thereof or recombinant expression vector, and a pharmaceutically acceptable formulation agent selected for suitability with the mode of administration. Suitable formulation materials preferably are non-toxic to recipients at the concentrations employed and can modify, maintain, or preserve, for example, the pH, osmolarity, viscosity, clarity, color, isotonicity, odor, sterility, stability, rate of dissolution or release, adsorption, or penetration of the composition. See Remington's Pharmaceutical Sciences (18th Ed., A. R. Gennaro, ed., Mack Publishing Company 1990).

The pharmaceutically active compounds (i.e., a polynucleotide, gene or region thereof or a vector) can be processed in accordance with conventional methods of pharmacy to produce medicinal agents for administration to patients, including humans and other mammals. Thus, the pharmaceutical composition comprising a polynucleotide, gene or region thereof or a recombinant expression vector may be made up in a solid form (including granules, powders or suppositories) or in a liquid form (e.g., solutions, suspensions, or emulsions).

The dosage regimen for treating a disease with a composition comprising a polynucleotide, gene or region thereof or expression vector is based on a variety of factors, including the type or severity of alcoholism, the age, weight, sex, medical condition of the patient, the route of administration, and the particular compound employed. Thus, the dosage regimen may vary widely, but can be determined routinely using standard methods. A typical dosage may range from about 0.1 mg/kg to about 100 mg/kg or more, depending on the factors mentioned above.

The frequency of dosing will depend upon the pharmacokinetic parameters of the polynucleotide, gene or region thereof or vector in the formulation being used. Typically, a clinician will administer the composition until a dosage is reached that achieves the desired effect. The composition may therefore be administered as a single dose, as two or more doses (which may or may not contain the same amount of the desired molecule) over time, or as a continuous infusion via implantation device or catheter. Further refinement of the appropriate dosage is routinely made by those of ordinary skill in the art and is within the ambit of tasks routinely performed by them. Appropriate dosages may be ascertained through use of appropriate dose-response data.

The cells of a mammalian subject may be transfected in vivo, ex vivo, or in vitro. Administration of a polynucleotide, gene or region thereof or a recombinant vector containing a polynucleotide, gene or region thereof to a target cell in vivo may be accomplished using any of a variety of techniques well known to those skilled in the art. For example, U.S. Pat. No. 5,672,344 describes an in vivo viral-mediated gene transfer system involving a recombinant neurotrophic HSV-1 vector. The above-described compositions of polynucleotides, genes and regions thereof and recombinant vectors can be transfected in vivo by oral, buccal, parenteral, rectal, or topical administration as well as by inhalation spray. The term “parenteral” as used herein includes, subcutaneous, intravenous, intramuscular, intrasternal, infusion techniques or intraperitoneally.

While the nucleic acids and/or vectors of the invention can be administered as the sole active pharmaceutical agent, they can also be used in combination with one or more vectors of the invention or other agents. When administered as a combination, the therapeutic agents can be formulated as separate compositions that are given at the same time or different times, or the therapeutic agents can be given as a single composition.

Another delivery system for polynucleotides or genes of the invention and regions thereof is a “non-viral” delivery system. Techniques that have been used or proposed for gene therapy include DNA-ligand complexes, adenovirus-ligand-DNA complexes, direct injection of DNA, CaPO₄ precipitation, gene gun techniques, electroporation, lipofection, and colloidal dispersion (Mulligan, R., (1993) Science, 260 (5110):926-32). Any of these methods are widely available to one skilled in the art and would be suitable for use in the present invention. Other suitable methods are available to one skilled in the art, and it is to be understood that the present invention may be accomplished using any of the available methods of transfection. Several such methodologies have been utilized by those skilled in the art with varying success. Id.

Where a polynucleotide or gene of the invention is up-regulated and exacerbates alcoholism, the expression of the polynucleotide or gene can be blocked or reduced or the level of the intact polypeptide product can be reduced in order to treat or prevent alcoholism or ameliorate symptoms associated with alcoholism, such as, for example, fatty liver, alcoholic hepatitis, fibrosis, cirrhosis, hypertension, weakened heart muscle, and arrhythmia. This can be accomplished by, for example, the use of antisense oligonucleotides, triple helix base pairing methodology or ribozymes. Alternatively, drugs or antibodies that bind to and inactivate the polypeptide product can be used.

Antisense oligonucleotides are nucleotide sequences that are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of gene products of the invention in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, (1994) Meth. Mol. Biol., 20:1-8; Sonveaux, (1994) Meth. Mol. Biol., 26:1-72; Uhlmann et al., (1990) Chem. Rev., 90:543-583.

Modifications of gene expression can be obtained by designing antisense oligonucleotides that will form duplexes to the control, 5′, or regulatory regions of a gene of the invention. Oligonucleotides derived from the transcription initiation site, e.g., between positions −10 and +10 from the start site, are preferred.

Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, Molecular and Immulogic Approaches, Futura Publishing Co., Mt. Kisco, N.Y., 1994). An antisense oligonucleotide also can be designed to block translation of mRNA by preventing the transcript from binding to ribosomes.

Precise complementarity is not required for successful complex formation between an antisense oligonucleotide and the complementary sequence of a polynucleotide. Antisense oligonucleotides that comprise, for example, 2, 3, 4, or 5 or more stretches of contiguous nucleotides that are precisely complementary to a polynucleotide, each separated by a stretch of contiguous nucleotides which are not complementary to adjacent nucleotides, can provide sufficient targeting specificity for mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular polynucleotide sequence.

Antisense oligonucleotides can be modified without affecting their ability to hybridize to a polynucleotide or gene of the invention or regions thereof. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′,5′-substituted oligonucleotide in which the 3′ hydroxyl group or the 5′ phosphate group are substituted, also can be employed in a modified antisense oligonucleotide. These modified oligonucleotides can be prepared by methods well known in the art. See, e.g., Agrawal et al., (1992) Trends Biotechnol., 10:152-158; Uhlmann et al., (1990) Chem. Rev., 90:543-584; Uhlmann et al., (1987) Tetrahedron. Lett., 215:3539-3542.

Ribozymes are RNA molecules with catalytic activity. See, e.g., Cech, (1987) Science, 236:1532-1539; Cech, (1990) Ann. Rev. Biochem., 59:543-568; Cech, (1992) Curr. Opin. Struct. Biol., 2:605-609; Couture & Stinchcomb, (1996) Trends Genet., 12:510-515. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of a polynucleotide or gene of the invention or a region thereof can be used to generate ribozymes that will specifically bind to mRNA transcribed from the polynucleotide. Methods of designing and constructing ribozymes which can cleave RNA molecules in trans in a highly sequence specific manner have been developed and described in the art (see Haseloff et al. (1988) Nature, 334:585-591). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, e.g., Gerlach et al., EP 321,201).

Specific ribozyme cleavage sites within a RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate RNA targets also can be evaluated by testing accessibility to hybridization with complementary oligonucleotides using ribonuclease protection assays. The nucleotide sequences shown in SEQ ID NOs:1-14, their complements and their corresponding genes and regions thereof provide sources of suitable hybridization region sequences. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease polynucleotide expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells.

As taught in Haseloff et al., U.S. Pat. No. 5,641,673, ribozymes can be engineered so that ribozyme expression will occur in response to factors that induce expression of a target gene. Ribozymes also can be engineered to provide an additional level of regulation, so that destruction of mRNA occurs only when both a ribozyme and a target gene are induced in the cells.

Polypeptides or antibodies to the polypeptides of the invention can also be used directly as therapeutics to prevent, treat, modulate, or ameliorate disease. The mammalian subject (preferably a human) can be given a recombinant or synthetic form of the polypeptide in one of many possible different formulations, including, but not limited to, subcutaneous, intravenous, intramuscular, intraperitoneal, or intracranial injections of a solution of the polypeptide or antibody, or a suspension of a crystallized form of the polypeptide or antibody; topical creams or slow release cutaneous patch containing the polypeptide; encapsulated forms for oral or other gastrointestinal delivery of the polypeptide or antibody. In some cases, delivery of the polypeptide or antibody may be in the form of injection or transplantation of cells or tissues containing an expression vector such that a recombinant form of the polypeptide will be secreted by the cells or tissues, as described above for transfected cells.

The frequency and dosage of the administration of the polypeptides or antibodies will be determined by factors such as the biological activity of the pharmacological preparation, the persistence and clearance of the active protein, and the goals of treatment. In the case of antibody therapies, the frequency and dosage will also depend on the ability of the antibody to bind and neutralize the target molecules in the target tissues.

Diagnostic Tests

Pathological conditions or susceptibility to pathological conditions, such as alcoholism, can be diagnosed using methods of the invention. Testing for expression of a polynucleotide or gene of the invention or regions thereof or for the presence of the polynucleotide or gene product can correlate with the severity of the alcoholism and can also indicate appropriate treatment. Furthermore, testing for regulation of a polynucleotide or gene of the invention or regions thereof or a panel of polynucleotides or genes of the invention or regions thereof can be used in drug development studies to assess the efficacy or toxicity of any experimental therapeutic. For example, the presence of a mutation in a polynucleotide or gene of the invention or regions thereof can be determined through sequencing techniques known to those skilled in the art and alcoholism or a susceptibility to alcoholism can be diagnosed based on the presence of the mutation. Further, an alteration in expression of a polypeptide encoded by a polynucleotide or gene of the invention can be detected, where the presence of an alteration in expression of the polypeptide is indicative of alcoholism or susceptibility to alcoholism. The alteration in expression can be an increase in the amount of expression or a decrease in the amount of expression, i.e. a modulation in expression. For example, DST CAR1_{—}3 expression decreases in the alcohol preferring rats as a result of polymorphisms in the coding and 3′ untranslated regions of the mRNA. This polymorphism causes a decrease in expression of both the mRNA and protein. This polymorphism can therefore be used as a diagnostic test for susceptibility to alcoholism or alcohol seeking behavior.

The use of diagnostic tests is not limited to determining the presence of or susceptibility to alcoholism. In many cases, the diagnostic test can be used to assess stage of alcoholism, especially in situations where such an objective lab test has no alternative reliable subjective test available. These tests can be used to follow the course of alcoholism, help predict the future course of alcoholism, or determine the possible reversal of alcoholism. For example, the level of expression of polynucleotides, genes, polypeptides of the invention or regions thereof may be indicative of the stage of alcoholism or progression of alcoholism.

In drug development studies, these tests can be useful as efficacy markers, so that the ability of any new therapeutics to treat alcoholism can be evaluated on the basis of these objective assays. The utility of these diagnostic tests will first be determined by developing statistical information correlating the specific lab test values with several clinical parameters so that the lab test values can be known to reliably predict certain clinical conditions.

In many cases, the diagnostic lab tests based on the polynucleotides, genes, antibodies or polypeptides of the invention, i.e., gene expression profiles of polynucleotides or polypeptides encoded by the polynucleotides identified in SEQ ID NOs: 1-14, will be important markers of drug or disease toxicity. The markers of toxicity versus drug efficacy will be determined by studies correlating the effects of known toxins or pathological conditions with specific alterations in gene regulation. Toxicity markers generated in this fashion will be useful to distinguish the various therapeutic versus deleterious effects on cells or tissues in the patient.

As an additional method of diagnosis, a first biological sample from a patient suspected of having alcoholism, is obtained along with a second sample from a suitable comparable control source. A biological sample can comprise saliva, blood, cerebrospinal fluid, amniotic fluid, urine, feces, tissue, or the like. A suitable control source can be obtained from one or more mammalian subjects that do not have alcoholism. For example, the average concentration and distribution of a polynucleotide, gene, or polypeptide of the invention or a region thereof can be determined from biological samples taken from a representative population of mammalian subjects, wherein the mammalian subjects are the same species as the subject from which the test sample was obtained. The amount of at least one polypeptide, gene, polynucleotide of the invention or region thereof is determined in the first and second sample. The amounts of the polypeptide in the first and second samples are compared. A patient is diagnosed as having alcoholism, if the amount of the polypeptide, gene, polynucleotide of the invention or a region thereof in the first sample is greater than or less than the amount of the polypeptide, gene, polynucleotide of the invention or a region thereof in the second sample. Preferably, the amount of polypeptide, gene, polynucleotide of the invention or a region thereof in the first sample falls in the range of samples taken from a representative group of patients with alcoholism.

The method for diagnosing a pathological condition can comprise a step of detecting nucleic acid molecules comprising a nucleotide sequence in a panel of at least two nucleotide sequences, wherein at least one sequence in said panel is at least 95% identical to a sequence of at least 50 contiguous nucleotides in a sequence selected from said group.

The present invention also includes a diagnostic system, preferably in kit form, for assaying for the presence of the polypeptide of the present invention in a body sample, including, but not limited to brain, cell suspensions or tissue sections; or a body fluid sample, such as CSF, blood, plasma or serum, where it is desirable to detect the presence, and preferably the amount, of the polypeptide of this invention in the sample according to the diagnostic methods described herein.

In a related embodiment, the discovery of differential expression patterns for the molecules of the invention allows for screening of test compounds with an eye to modulating a particular expression pattern; for example, screening can be done for compounds that will convert an expression profile for a poor prognosis to a better prognosis. These methods can also be done on the protein basis; that is, protein expression levels of the molecules of the invention, such as, for example, polypeptides encoded by the polynucleotides identified in SEQ ID NOs: 1-14, can be evaluated for diagnostic and prognostic purposes or to screen test compounds.

In addition, the invention provides methods of conducting high-throughput screening for test compounds capable of inhibiting activity of proteins encoded by the polynucleotides of the invention, i.e., SEQ ID NOs: 1-14. The method of high-throughput screening involves combining test compounds and the polypeptide and measuring an effect of the test compound on the encoded polypeptide. Functional assays such as cytosensor microphysiometer, calcium flux assays such as FLIPR (Molecular Devices Corp, Sunnyvale, Calif.), or the TUNEL assay may be employed to measure cellular activity.

The invention also provides a method of screening test compounds for inhibitors of alcoholism and the pharmaceutical compositions comprising the test compounds. The method for screening comprises obtaining samples from subjects afflicted with alcoholism, maintaining separate aliquots of the samples with a plurality of test compounds, and comparing expression of a molecules of the invention, i.e., SEQ ID NOs: 1-14, in each of the aliquots to determine whether any of the test compounds provides a substantially modulated level of expression relative to samples with other test compounds or to an untreated sample. In addition, methods of screening may be devised by combining a test compound with a protein and thereby determining the effect of the test compound on the polypeptide.

In a related embodiment, a nucleic acid molecule can be used as a probe (i.e., an oligonucleotide) to detect the presence of a polynucleotide of the present invention, a gene corresponding to a polynucleotide of the present invention or a region thereof, or a mRNA in a cell that is diagnostic for the presence or expression of a polypeptide of the present invention in the cell. The nucleic acid molecule probes can be of a variety of lengths from at least about 10 to about 5000 nucleotides long, although they will typically be about 20 to 500 nucleotides in length. The probe can be used to detect the polynucleotide, gene, gene region or mRNA through hybridization methods that are well known in the art.

In a related embodiment, detection of genes corresponding to the polynucleotides of the present invention can be conducted by primer extension reactions, such as polymerase chain reaction (PCR). To that end, PCR primers are utilized in pairs, as is well known, based on the nucleotide sequence of the gene to be detected. Preferably, the nucleotide sequence is a portion of the nucleotide sequence of a polynucleotide of the present invention. Particularly preferred PCR primers can be derived from any portion of a DNA sequence encoding a polypeptide of the present invention, but are preferentially from regions that are not conserved in other cellular proteins.

Preferred PCR primer pairs useful for detecting the genes corresponding to the polynucleotides of the present invention and expression of these genes are described below. Nucleotide primers from the corresponding region of the polypeptides of the present invention described herein are readily prepared and used as PCR primers for detection of the presence or expression of the corresponding gene in any of a variety of tissues.

In another embodiment, a diagnostic system, preferably in kit form, is contemplated for assaying for the presence of the polypeptide of the present invention or an antibody immunoreactive with the polypeptide of the present invention in a body fluid sample. Such diagnostic kits are useful for monitoring the fate of a therapeutically administered polypeptide of the present invention or an antibody immunoreactive with the polypeptide of the present invention. The system includes, in an amount sufficient for at least one assay, a polypeptide of the present invention and/or a subject antibody as a separately packaged immunochemical reagent. Instructions for use of the packaged reagent(s) are also typically included.

A diagnostic system of the present invention preferably also includes a label or indicating means capable of signaling the formation of an immunocomplex containing a polypeptide or antibody molecule of the present invention.

Any label or indicating means can be linked to or incorporated in an expressed protein, polypeptide, or antibody molecule that is part of an antibody or monoclonal antibody composition of the present invention or used separately, and those atoms or molecules can be used alone or in conjunction with additional reagents. Such labels are themselves well-known in clinical diagnostic chemistry and constitute a part of this invention only insofar as they are utilized with otherwise novel proteins methods and/or systems.

The labeling means can be a fluorescent labeling agent that chemically binds to antibodies or antigens without denaturing them to form a fluorochrome (dye) that is a useful immunofluorescent tracer. Suitable fluorescent labeling agents are fluorochromes such as fluorescein isocyanate (FIC), fluorescein isothiocyanate (FITC), 5-dimethylamine-1-naphthalenesulfonyl chloride (DANSC), tetramethylrhodamine isothiocyanate (TRITC), lissamine, rhodamine 8200 sulphonyl chloride (RB 200 SC) and the like. A description of immunofluorescence analysis techniques is found in DeLuca, “Immunofluorescence Analysis”, in Antibody As a Tool, Marchalonis et al., Eds., John Wiley & Sons, Ltd., pp. 189-231 (1982), which is incorporated herein by reference. Other suitable labeling agents are known to those skilled in the art.

In preferred embodiments, the indicating group is an enzyme, such as horseradish peroxidase (HRP), glucose oxidase, or the like. In such cases where the principal indicating group is an enzyme such as HRP or glucose oxidase, additional reagents are required to visualize the formation of the receptor-ligand complex. Such additional reagents for HRP include hydrogen peroxide and an oxidation dye precursor, such as diaminobenzidine. Such additional reagents for biotin include streptavidin. An additional reagent useful with glucose oxidase is 2,2′-amino-di-(3-ethyl-benzthiazoline-G-sulfonic acid) (ABTS).

Radioactive elements are also useful labeling agents and are used illustratively herein. An exemplary radiolabeling agent is a radioactive element that produces gamma ray emissions. Elements which themselves emit gamma rays, such as ¹²⁴I, ¹²⁵I, ¹²⁸I, ¹³²I and ⁵¹Cr represent one class of gamma ray emission-producing radioactive element indicating groups. Particularly preferred is ¹²⁵I. Another group of useful labeling means are those elements such as ¹¹C, ¹⁸F, ¹⁵O and ¹³N which themselves emit positrons. The positrons so emitted produce gamma rays upon encounters with electrons present in the animal's body. Also useful is a beta emitter, such ¹¹¹indium or ³H.

The linking of labels or labeling of polypeptides and proteins is well known in the art. For instance, antibody molecules produced by a hybridoma can be labeled by metabolic incorporation of radioisotope-containing amino acids provided as a component in the culture medium (see, e.g., Galfre et al., Meth. Enzymol., 73:3-46 (1981)). The techniques of protein conjugation or coupling through activated functional groups are particularly applicable (see, e.g., Aurameas, et al., Scand. J. Immunol., Vol. 8 Suppl. 7:7-23 (1978); Rodwell et al., Biotech., 3:889-894 (1984); and U.S. Pat. No. 4,493,795).

The diagnostic systems can also include, preferably as a separate package, a specific binding agent. Exemplary specific binding agents are second antibody molecules, complement proteins or fragments thereof, such as, S. aureus protein A, and the like. Preferably the specific binding agent binds the reagent species when that species is present as part of a complex.

In preferred embodiments, the specific binding agent is labeled. However, when the diagnostic system includes a specific binding agent that is not labeled, the agent is typically used as an amplifying means or reagent. In these embodiments, the labeled specific binding agent is capable of specifically binding the amplifying means when the amplifying means is bound to a reagent species-containing complex.

The diagnostic kits of the present invention can be used in an “ELISA” format to detect the quantity of the polypeptide of the present invention in a sample. A description of the ELISA technique is found in Sites et al., Basic and Clinical Immunology, 4th Ed., Chap. 22, Lange Medical Publications, Los Altos, Calif. (1982) and in U.S. Pat. No. 3,654,090; U.S. Pat. No. 3,850,752; and U.S. Pat. No. 4,016,043, which are all incorporated herein by reference.

Thus, in some embodiments, a polypeptide of the present invention, an antibody or a monoclonal antibody of the present invention can be affixed to a solid matrix to form a solid support that comprises a package in the subject diagnostic systems.

A reagent is typically affixed to a solid matrix by adsorption from an aqueous medium, although other modes of affixation applicable to proteins and polypeptides can be used that are well known to those skilled in the art. Exemplary adsorption methods are described herein.

Useful solid matrices are also well known in the art. Such materials are water insoluble and include the cross-linked dextran available under the trademark SEPHADEX from Pharmacia Fine Chemicals (Piscataway, N.J.), agarose, polystyrene beads of about 1 micron (μm) to about 5 millimeters (mm) in diameter available from several suppliers (e.g., Abbott Laboratories, Chicago, Ill.), polyvinyl chloride, polystyrene, cross-linked polyacrylamide, nitrocellulose- or nylon-based webs (sheets, strips or paddles) or tubes, plates or the wells of a microtiter plate, such as those made from polystyrene or polyvinylchloride.

The reagent species, labeled specific binding agent, or amplifying reagent of any diagnostic system described herein can be provided in solution, as a liquid dispersion or as a substantially dry power, e.g., in lyophilized form. Where the indicating means is an enzyme, the enzyme's substrate can also be provided in a separate package of a system. A solid support such as the before-described microtiter plate and one or more buffers can also be included as separately packaged elements in this diagnostic assay system.

The packaging materials discussed herein in relation to diagnostic systems are those customarily utilized in diagnostic systems.

Genes

The present invention also relates to the genes corresponding to SEQ ID NOs:1-14, and the polypeptides encoded by the polynucleotides or genes or regions thereof of SEQ ID NOs:1-14. The corresponding gene can be isolated in accordance with known methods using the sequence information disclosed herein. Such methods include preparing probes or primers from the disclosed sequence and identifying or amplifying the corresponding gene from appropriate sources of genomic material.

Homologs, Paralogs and Orthologs

Also provided in the present invention are homologs of the polynucleotides, polypeptides and genes of the invention and regions thereof, including paralogous genes and orthologous genes. Nucleic acid homologs may be isolated and identified using suitable probes or primers from the sequences provided herein and screening a suitable nucleic acid source for the desired homolog. Studies of gene and protein evolution often involve the comparison of homologs, which are sequences that have common origins but may or may not have common activity. Sequences that share an arbitrary level of similarity determined by alignment of matching bases are called homologous.

There are many cases in which genes have duplicated, assumed somewhat different functions and been moved to other regions of the genome (e.g., alpha and beta globin). Such related genes in the same species are referred to as paralogs (e.g., Lundin, 1993, who refers to Fitch, 1976 for this distinction). They must be distinguished from orthologs (homologous genes in different species, such as beta globin in human and mouse) if any sensible comparisons are to be made. These terms as relate to genes are formally defined as follows:

As used herein, “paralogous genes” are genes within the same species produced by gene duplication in the course of evolution. They may be arranged in clusters or distributed on different chromosomes, an arrangement which is usually conserved in a wide range of vertebrates.

As used herein, “orthologous genes” describes homologous genes in different species that are descended from the same gene in the nearest common ancestor. Orthologs tend to have similar function.

In reports of previous Human Gene Mapping Workshops, the Comparative Gene Mapping Committee recommended explicit criteria for establishing homology between genes mapped in different species, as well as urging inclusion of specific criteria in comparative gene mapping publications (O'Brien and Graves, 1991). The evidence for gene homology might also be recorded in The Comparative Animal Genome database (TCAGdb). Revised criteria for determining homology can include any of the following (the most stringent are shown with asterisks):

Gene or Other Nucleotide Sequence:

similar nucleotide sequence*

cross-hybridization to the same molecular-probe*

conserved map position*

Protein or Polypeptide:

similar amino acid sequence*

similar subunit structure and formation of functional heteropolymer

immunological cross-reaction

similar expression profile

similar subcellular location

similar substrate specificity

similar response to specific inhibitors

Phenotype:

similar mutant phenotype

complementation of function*

Two new criteria have recently been added. Because of the accumulation of overwhelming evidence for linkage conservation among mammal and vertebrate species, conserved map position may now itself constitute an important criterion of homology, and is particularly valuable in distinguishing between members of a gene family. Complementation of function has also been added, because it is now possible to establish complementation of function by transfection across even the widest species barriers.

More recent studies have also demonstrated that some of these criteria are much more stringent than others. A strong basis for homology would be a demonstration of high DNA or amino acid sequence similarity, plus in addition to conservation of map position between flanking homologous markers. Less robust immunological and biochemical criteria for gene homology would need to be confirmed at least by gene position. The assumption of gene homology must be considered a working hypothesis, and may later be further confirmed when further scientific criteria are applied.

Preferred embodiments of the present invention include homologs, paralogs and orthologs of the polynucleotides, polypeptides and genes of the invention and regions thereof.

Polypeptides

The polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art. See, e.g., Curr. Prot. Mol. Bio., Chapter 16.

The polypeptides may be in the form of the secreted protein, including the mature form, or may be a part of a larger protein, such as a fusion protein (see below). It is often advantageous to include an additional amino acid sequence that contains secretory or leader sequences, pro-sequences, sequences which aid in purification (such as multiple histidine residues), or an additional sequence for stability during recombinant production.

The polypeptides of the present invention are preferably provided in an isolated form, and preferably are substantially purified. A recombinantly produced version of a polypeptide, including the secreted polypeptide, can be substantially purified by the one-step method described in Smith & Johnson (Gene, 67:31-40, 1988). Polypeptides of the invention also can be purified from natural or recombinant sources using antibodies of the invention raised against the secreted protein according to methods that are well known in the art.

Signal Sequences

Methods for predicting whether a protein has a signal sequence, as well as the cleavage point for that sequence, are available. For instance, the method of McGeoch uses the information from a short N-terminal charged region and a subsequent uncharged region of the complete (uncleaved) protein (Virus Res., 3:271-286 (1985)). The method of von Heinje uses the information from the residues surrounding the cleavage site, typically residues −13 to +2, where +1 indicates the amino terminus of the secreted protein (Nucleic Acids Res., 14:4683-4690 (1986)). Therefore, from a deduced amino acid sequence, a signal sequence and mature sequence can be identified.

The deduced amino acid sequence of a secreted polypeptide can be analyzed by a computer program called Signal P (Nielsen et al., Protein Engineering, 10:1-6 (1997), which predicts the cellular location of a protein based on the amino acid sequence. As part of this computational prediction of localization, the methods of McGeoch and von Heinje are incorporated.

As one of ordinary skill in the art will appreciate, however, cleavage sites sometimes vary from organism to organism and cannot be predicted with absolute certainty. Accordingly, the present invention provides secreted polypeptides having a sequence corresponding to the translations of SEQ ID NOs:1-14 and their corresponding genes which have an N-terminus beginning within 5 residues (i.e., + or −5 residues) of the predicted cleavage point. Similarly, it is also recognized that in some cases, cleavage of the signal sequence from a secreted protein is not entirely uniform, resulting in more than one secreted species. These polypeptides, and the polynucleotides and genes encoding such polypeptides, are contemplated by the present invention.

Moreover, the signal sequence identified by the above analysis may not necessarily predict the naturally occurring signal sequence. For example, the naturally occurring signal sequence may be further upstream from the predicted signal sequence. However, it is likely that the predicted signal sequence will be capable of directing the secreted protein to the ER. These polypeptides, and the polynucleotides and genes encoding such polypeptides, are contemplated by the present invention.

Polynucleotide, Polypeptide and Gene Variants

Polynucleotide, polypeptide and gene variants differ from the polynucleotides, polypeptides and genes of the present invention, but retain essential properties thereof. In general, variants have close similarity overall and are identical in many regions to the polynucleotide or polypeptide of the present invention.

Further embodiments of the present invention include polynucleotides having at least 80% identity, more preferably at least 90% identity, and most preferably at least 95%, 96%, 97%, 98% or 99% identity to a sequence contained in SEQ ID NOs:1-14. Of course, due to the degeneracy of the genetic code, one of ordinary skill in the art will immediately recognize that a large number of the polynucleotides having at least 85%, 90%, 95%, 96%, 97%, 980%, or 99% identity, polynucleotides at least ten bases in length hybridizable to polynucleotide comprising at least one of SEQ ID NOs: 1-14, polynucleotides comprising at least one SEQ ID NOs: 1-14 with sequential nucleotide deletions from either the 5′ terminus or the 3′ terminus, or a species homolog of polynucleotides comprising at least one of SEQ ID NOs: 1-14 will encode a polypeptide identical to an amino acid sequence contained in the translations of SEQ ID NOs:1-14.

Further embodiments of the present invention include genes and regions thereof having at least 80% identity, more preferably at 90% identity, and most preferably at least 95%, 96%, 97%, 98% or 99% identity to genes corresponding to a sequence contained in SEQ ID NOs:1-14 and regions thereof. Of course, due to the degeneracy of the genetic code, one of ordinary skill in the art will immediately recognize that a large number of the genes having at least 85%, 90%, 95%, 96%, 97%, or 99% identity respectively to genes of the invention, genes hybridizable to genes of the invention, genes of the invention with sequential nucleotide deletions from either the 5′ terminus or the 3′ terminus, or a species homolog of genes of the invention will encode a polypeptide identical to an amino acid sequence contained in the translations of genes of the invention.

Further embodiments of the present invention also include polypeptides having at least 80% identity, more preferably at least 85% identity, more preferably at least 90% identity, and most preferably at least 95%, 96%, 97%, 98% or 99% identity to an amino acid sequence contained in translations of SEQ ID NOs:1-14 and their corresponding genes. Preferably, the above polypeptides should exhibit at least one biological activity of the protein. In a preferred embodiment, polypeptides of the present invention include polypeptides having at least 90% similarity, more preferably at least 95% similarity, and still more preferably at least 96%, 97%, 98%, or 99% similarity to an amino acid sequence contained in translations of SEQ ID NOs:1-14 and their corresponding genes. Methods for aligning polynucleotides, polypeptides, genes or regions thereof are codified in computer programs, including the GCG program package (Devereux et al., Nuc. Acids Res. 12:387 (1984)), BLASTP, BLASTN, FASTA (Atschul et al., J. Molec. Biol. 215:403 (1990)), and Bestfit program (Wisconsin Sequence Analysis Package, Version 8 for Unix, Genetics Computer Group, University Research Park, 575 Science Drive, Madison, Wis. 53711) which uses the local homology algorithm of Smith and Waterman (Adv. in App. Math., 2:482-489 (1981)).

When using any of the sequence alignment programs to determine whether a particular sequence is, for example, 95% identical to a reference sequence, the parameters are set such that the percentage of identity is calculated over the full length of the reference polynucleotide or gene that gaps in identity of up to 5% of the total number of nucleotides in the reference polynucleotide or gene are allowed.

A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci., 6:237-245 (1990)). The term “sequence” includes nucleotide and amino acid sequences. In a sequence alignment the query and subject sequences are either both nucleotide sequences or both amino acid sequences. The result of said global sequence alignment is presented in terms of percent identity. Preferred parameters used in a FASTDB search of a DNA sequence to calculate percent identity are: Matrix=Unitary, k-tuple=4, Mismatch Penalty=1, Joining Penalty=30, Randomization Group Length=0, and Cutoff Score=1, Gap Penalty=5, Gap Size Penalty=0.05, and Window Size=500 or query sequence length in nucleotide bases, whichever is shorter. Preferred parameters employed to calculate percent identity and similarity of an amino acid alignment are: Matrix=PAM 150, k-tuple=2, Mismatch Penalty=1, Joining Penalty=20, Randomization Group Length=0, Cutoff Score=1, Gap Penalty=5, Gap Size Penalty=0.05, and Window Size=500 or query sequence length in amino acid residues, whichever is shorter.

For example, a polynucleotide having a nucleotide sequence of at least 95% “identity” to a sequence contained in SEQ ID NOs:1-14 means that the polynucleotide is identical to a sequence contained in SEQ ID NOs:1-14 or the cDNA except that the polynucleotide sequence may include up to five point mutations per each 100 nucleotides of the total length (not just within a given 100 nucleotide stretch). In other words, to obtain a polynucleotide having a nucleotide sequence at least 95% identical to SEQ ID NOs:1-14, up to 5% of the nucleotides in the sequence contained in SEQ ID NOs:1-14 or the cDNA can be deleted, inserted, or substituted with other nucleotides. These changes may occur anywhere throughout the polynucleotide.

Similarly, a polypeptide having an amino acid sequence having at least, for example, 95% “identity” to a reference polypeptide, means that the amino acid sequence of the polypeptide is identical to the reference polypeptide except that the polypeptide sequence may include up to five amino acid alterations per each 100 amino acids of the total length of the reference polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

The variants may contain alterations in the coding regions, non-coding regions, or both. Especially preferred are polynucleotide variants containing alterations that produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. Polynucleotide variants can be produced for a variety of reasons. For instance, a polynucleotide variant may be produced to optimize codon expression for a particular host (i.e., codons in the human mRNA may be changed to those preferred by a bacterial host, such as E. coli). Variants may also arise by the process of ribosomal frameshifting, by translational read-through at naturally occurring stop codons, and by decoding of in-frame translational stop codons UGA through insertion of selanocysteine (See The RNA World, 2^{nd} edition, ed: Gesteland, R. F., Cech, T. R., & Atkins, J. F.; Cold Spring Harbor Laboratory Press, 1999).

The variants may be allelic variants. Naturally occurring variants are called “allelic variants,” and refer to one of several alternate forms of a gene occupying a given locus on a chromosome of an organism (Lewin, Ed., Genes II, John Wiley & Sons, New York (1985)). These allelic variants can vary at either the polynucleotide and/or polypeptide level. Alternatively, non-naturally occurring variants may be produced by mutagenesis techniques or by direct synthesis. See, e.g., Curr. Prot. Mol. Bio., Chapter 8.

Using known methods of protein engineering and recombinant DNA technology, variants may be generated to improve or alter the characteristics of the polypeptides of the present invention. For example, polypeptide variants containing amino acid substitutions of charged amino acids with other charged or neutral amino acids may produce proteins with improved characteristics, such as decreased aggregation. As known, aggregation of pharmaceutical formulations both reduces activity and increases clearance due to the aggregate's immunogenic activity (see, e.g., Pinckard et al., Clin. Exp. Immunol. 2:331-340 (1967); Robbins et al., Diabetes, 36: 838-845 (1987); Cleland et al., Crit. Rev. Therap. Drug Carrier Sys., 10:307-377 (1993)). Similarly, interferon gamma exhibited up to ten times higher activity after deleting 8-10 amino acid residues from the carboxy terminus of this protein (Dobeli et al., J. Biotechnology, 7:199-216 (1988)).

Moreover, ample evidence demonstrates that variants often retain a biological activity similar to that of the naturally occurring protein. For example, Gayle et al. conducted extensive mutational analysis of human cytokine IL-1a (J. Biol. Chem., 268:22105-22111 (1993)). These investigators used random mutagenesis to generate over 3,500 individual IL-1 a mutants that averaged 2.5 amino acid changes per variant over the entire length of the molecule. Multiple mutations were examined at every possible amino acid position. The investigators concluded that “most of the molecule could be altered with little effect on either binding or biological activity.” In fact, only 23 unique amino acid sequences, out of more than 3,500 amino acid sequences examined, produced a protein that differed significantly in activity from the wild-type sequence. Another experiment demonstrated that one or more amino acids can be deleted from the N-terminus or C-terminus of the secreted protein without substantial loss of biological function. Ron et al. reported variant KGF proteins having heparin binding activity even after deleting 3, 8, or 27 amino-terminal amino acid residues (J. Biol. Chem. 268: 2984-2988 (1993)).

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes polypeptide variants that show substantial biological activity. Such variants include deletions, insertions, inversions, repeats, frameshifting, read-through translational variants, and substitutions selected according to general rules known in the art so as have little effect on activity. For example, guidance concerning how to make phenotypically silent amino acid substitutions is provided in Bowie et al., Science, 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, the amino acid positions that have been conserved between species can be identified. These conserved amino acids are likely important for protein function. In contrast, the amino acid positions in which substitutions have been tolerated by natural selection indicate positions which are not critical for protein function. Thus, positions tolerating amino acid substitution may be modified while still maintaining biological activity of the protein.

The second strategy uses genetic engineering to introduce amino acid changes at specific positions of a cloned gene to identify regions critical for protein function. For example, site-directed mutagenesis or alanine-scanning mutagenesis (the introduction of single alanine mutations at every residue in the molecule) can be used (Cunningham et al., Science, 244:1081-1085 (1989)). The resulting mutant molecules can then be tested for biological activity.

According to Bowie et al., these two strategies have revealed that proteins are surprisingly tolerant of amino acid substitutions. The authors further indicate which amino acid changes are likely to be permissive at certain amino acid positions in the protein. For example, the most buried or interior (within the tertiary structure of the protein) amino acid residues require nonpolar side chains, whereas few features of surface or exterior side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln; replacement of the basic residues Lys, Arg, and His; replacement of the aromatic residues Phe, Tyr, and Trp; and replacement of the small-sized amino acids Ala, Ser, Thr, Met, and Gly.

Besides conservative amino acid substitution, variants of the present invention include:

(i) substitutions with one or more of the non-conserved amino acid residues, where the substituted amino acid residues may or may not be one encoded by the genetic code;

(ii) substitution with one or more of amino acid residues having a substituent group; (iii) fusion of the mature polypeptide with another compound, such as a compound to increase the stability and/or solubility of the polypeptide (e.g., polyethylene glycol); (iv) fusion of the polypeptide with additional amino acids, such as an IgG Fc fusion region peptide, a leader or secretory sequence, or a sequence facilitating purification. Such variant polypeptides are deemed to be within the scope of those skilled in the art from the teachings herein.

Polynucleotide and Polypeptide Fragments

In the present invention, a “polynucleotide fragment” and “region of a gene” refers to a short polynucleotide having a nucleic acid sequence contained in SEQ ID NOs:1-14. The short nucleotide fragments are preferably at least about 15 nucleotides (nt), and more preferably at least about 20 nt, still more preferably at least about 30 nt, and even more preferably, at least about 40 nt in length. A fragment “at least 20 nt in length,” for example, is intended to include 20 or more contiguous bases from the cDNA sequence contained in that shown in SEQ ID NOs:1-14. These nucleotide fragments are useful as diagnostic probes and primers as discussed herein. Of course, larger fragments (e.g., 50, 150, and greater than 150 nucleotides) are preferred.

Moreover, representative examples of polynucleotide fragments of the invention, include, for example, fragments having a sequence from about nucleotide number 1-50, 51-100, 101-140, 151-200,201-250, 251-300, 301-350, 351-400, 401-450, to the end of SEQ ID NOs:1-14. In this context “about” includes the particularly recited ranges, larger or smaller by several nucleotides (i.e., 5, 4, 3, 2, or 1 nt) at either terminus or at both termini. Preferably, these fragments encode a polypeptide that has biological activity.

In the present invention, a “polypeptide fragment” refers to a short amino acid sequence contained in the translations of SEQ ID NOs:1-14. Protein fragments may be “free-standing,” or comprised within a larger polypeptide of which the fragment forms a part or region, most preferably as a single continuous region. Representative examples of polypeptide fragments of the invention include, for example, fragments from about amino acid number 1-20, 21-40, 41-60, or 61 to the end of the coding region. Moreover, polypeptide fragments can be about 20, 30, 40, 50, or 60 amino acids in length. In this context “about” includes the particularly recited ranges, larger or smaller by several amino acids (5, 4, 3, 2, or 1) at either extreme or at both extremes.

In situations where a DST of the present invention is not a translatable polypeptide, i.e., where the DST is in whole or in part of the 3′ untranslated region of its corresponding gene, the translation product or region of the translation product of the gene corresponding to the DST is intended to be encompassed by the terms “polypeptide” or “polypeptide fragment” as used herein.

Preferred polypeptide fragments include the secreted protein as well as the mature form. Further preferred polypeptide fragments include the secreted protein or the mature form having a continuous series of deleted residues from the amino or the carboxy terminus, or both. For example, any number of amino acids ranging from 1-60, can be deleted from the amino terminus of either the secreted polypeptide or the mature form. Similarly, any number of amino acids ranging from 1-30, can be deleted from the carboxy terminus of the secreted protein or mature form. Furthermore, any combination of the above amino and carboxy terminus deletions are preferred. Similarly, polynucleotide fragments encoding these polypeptide fragments are also preferred.

Also preferred are polypeptide and polynucleotide fragments characterized by structural or functional domains, such as fragments that comprise alpha-helix and alpha-helix-forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Polypeptide fragments of the translations of SEQ ID NOs:1-14 and their corresponding genes falling within conserved domains are specifically contemplated by the present invention. Moreover, polynucleotide fragments encoding these domains are also contemplated.

Other preferred fragments are biologically active fragments or the polynucleotide or gene encoding biologically active fragments. Biologically active fragments are those exhibiting activity similar, but not necessarily identical, to an activity of the polypeptide of the present invention. The biological activity of the fragments may include an improved desired activity, or a decreased undesirable activity.

Epitopes and Antibodies or Binding Partners to Them

Fragments which function as epitopes may be produced by any conventional means. (See, e.g., Houghten, R. A., Proc. Natl. Acad. Sci. USA, 82:5131-5135 (1985), further described in U.S. Pat. No. 4,631,211).

In the present invention, antigenic epitopes preferably contain a sequence of at least seven, more preferably at least nine, and most preferably between about 15 to about 30 amino acids. Antigenic epitopes are useful to raise antibodies, including monoclonal antibodies, which specifically bind the epitope. (See, e.g., Wilson et al., Cell, 37:767-778 (1984); Sutcliffe et al., Science, 219:660-666 (1983)).

Similarly, immunogenic epitopes can be used to induce antibodies or to select binding partners according to methods well known in the art. (See, e.g., Sutcliffe et al., (1983) supra; Wilson et al., (1984) supra; Chow et al., Proc. Natl. Acad. Sci., USA, 82:910-914; and Bittle et al., J. Gen. Virol., 66:2347-2354 (1985)). A preferred immunogenic epitope includes the secreted protein. The immunogenic epitope may be presented together with a carrier protein, such as an albumin, to an animal system (such as rabbit or mouse). Alternatively, the immunogenic epitope may be prescribed without a carrier, if the sequence is of sufficient length (at least about 25 amino acids). However, immunogenic epitopes comprising as few as 8 to 10 amino acids have been shown to be sufficient to raise antibodies capable of binding to, at the very least, linear epitopes in a denatured polypeptide (e.g., in Western blotting.)

As used herein, the term “antibody” (Ab) or “monoclonal antibody” (mAb) is meant to include intact molecules as well as antibody fragments (such as, for example, Fab and F(ab′)2 fragments) which are capable of specifically binding to protein. Fab and F(ab′)2 fragments lack the Fc fragment of intact antibody, clear more rapidly from the circulation, and may have less non-specific tissue binding than an intact antibody (Wahl et al., J. Nucl. Med., 24:316-325, 1983). Thus, these fragments are preferred, as well as the products of a Fab or other immunoglobulin expression library. Moreover, antibodies of the present invention include chimeric, single chain, and human and humanized antibodies.

The antibodies may be chimeric antibodies, e.g., humanized versions of murine monoclonal antibodies. Such humanized antibodies may be prepared by known techniques, and offer the advantage of reduced immunogenicity when the antibodies are administered to humans. See, e.g., Co et al., Nature, 351:501-2 (1991). In one embodiment, a humanized monoclonal antibody comprises the variable region of a murine antibody (or just the antigen binding site thereof) and a constant region derived from a human antibody. Alternatively, a humanized antibody fragment may comprise the antigen binding site of a murine monoclonal antibody and a variable region fragment (lacking the antigen-binding site) derived from a human antibody. Procedures for the production of chimeric and further engineered monoclonal antibodies include those described in Riechmann et al., Nature, 332:323, 1988, Liu et al., PNAS, 84:3439, 1987, Larrick et al., Bio/Technology, 7:934, 1989, and Winter and Harris, TIPS, 14:139, May, 1993, Zou et al., Science 262:1271-4, 1993, Zou et al., Curr. Biol., 4:1099-103, 1994, and Walls et al., Nucleic Acids Res., 21:2921-9, 1993.

One method for producing a human antibody comprises immunizing a non-human animal, such as a transgenic mouse, with a polypeptide translated from a nucleotide sequence chosen from SEQ ID NOs:1-14 or their corresponding genes, whereby antibodies directed against the polypeptide translated from a nucleotide sequence chosen from SEQ ID NOs:1-14 or their corresponding genes are generated in said animal. Procedures have been developed for generating human antibodies in non-human animals. The antibodies may be partially human, or preferably completely human. For example, mice have been prepared in which one or more endogenous immunoglobulin genes are inactivated by various means and human immunoglobulin genes are introduced into the mice to replace the inactivated mouse genes. Such transgenic mice may be genetically altered in a variety of ways. The genetic manipulation may result in human immunoglobulin polypeptide chains replacing endogenous immunoglobulin chains in at least some (preferably virtually all) antibodies produced by the animal upon immunization. Examples of techniques for production and use of such transgenic animals are described in U.S. Pat. Nos. 5,814,318, 5,569,825, and 5,545,806, which are incorporated by reference herein. Antibodies produced by immunizing transgenic animals with a polypeptide translated from a nucleotide sequence chosen from SEQ ID NOs:1-14 or their corresponding genes and methods of using such antibodies are provided herein.

Monoclonal antibodies may be produced by conventional procedures, e.g., by immortalizing spleen cells harvested from the transgenic animal after completion of the immunization schedule. The spleen cells may be fused with myeloma cells to produce hybridomas by conventional procedures. Examples of such techniques are described in U.S. Pat. No. 4,196,265, which is incorporated by reference herein.

A method for producing a hybridoma cell line comprises immunizing such a transgenic animal with an immunogen comprising at least seven contiguous amino acid residues of a polypeptide translated from a nucleotide sequence chosen from SEQ ID NOs:1-14 or their corresponding genes; harvesting spleen cells from the immunized animal; fusing the harvested spleen cells to a myeloma cell line, thereby generating hybridoma cells; and identifying a hybridoma cell line that produces a monoclonal antibody that binds a polypeptide translated from a nucleotide sequence chosen from SEQ ID NOs:1-14 or their corresponding genes. Such hybridoma cell lines, and monoclonal antibodies produced therefrom, are encompassed by the present invention. Monoclonal antibodies secreted by the hybridoma cell line are purified by conventional techniques. Examples of such techniques are described in U.S. Pat. No. 4,469,630 and U.S. Pat. No. 4,361,549.

Antibodies are only one example of binding partners to epitopes or receptor molecules. Other examples include, but are not limited to, synthetic peptides, which can be selected as a binding partner to an epitope or receptor molecule. The peptide may be selected from a peptide library as described by Appel et al., Biotechniques, 13, 901-905; and Dooley et al., J. Biol. Chem. 273, 18848-18856, 1998.

Binding assays can select for those binding partners (antibody, synthetic peptide, or other molecule) with highest affinity for the epitope or receptor molecule, using methods known in the art. Such assays may be done by immobilizing the epitope or receptor on a solid support, allowing binding of the library of antibodies or other molecules, and washing away those molecules with little or no affinity. Those binding partners or antibodies with highest affinity for the epitope or receptor will remain bound to the solid support. Alternatively, arrays of candidate binding partners may be immobilized, and a labeled soluble receptor molecule is allowed to interact with the array, followed by washing unbound receptors. High affinity binding is detectable by the presence of bound label.

Antibodies or other binding partners may be employed in an in vitro procedure, or administered in vivo to inhibit biological activity induced by a polypeptide translated from a nucleotide sequence chosen from SEQ ID NOs:1-14 or their corresponding genes. Disorders caused or exacerbated (directly or indirectly) by the interaction of such polypeptides of the present invention with cell surface receptors thus may be treated. A therapeutic method involves in vivo administration of a blocking antibody to a mammal in an amount effective for reducing a biological activity induced by a polypeptide translated from a nucleotide sequence chosen from SEQ ID NOs:1-14 or their corresponding genes.

Generally, antibodies or binding partners to receptors or cell surface polypeptides also can be linked to moieties, such as, for example, drug-loaded particles, antigens, DNA vaccines, immune modulators, other peptides, proteins for specific binding, and the like to the cells for targeting and enhanced delivery of the drug-loaded particles, antigens, DNA vaccines, immune modulators, other peptides, proteins for specific binding, and the like. Exemplary vaccines that can be specifically targeted to particular cells include, but are not limited to, rotavirus, influenza, diptheria, tetanus, pertussis, Hepatitis A, B and C, as well as conjugate vaccines, including S. pneumonia. Similarly, exemplary drugs that may be specifically targeted to particular cells include, but are not limited to, insulin, LHRH, buserlein, vasopressin and recombinant interleukins, such as IL-2 and IL-12. Additionally, exemplary vectors, such as, for example, adeno-associated virus, canarypox virus, adenovirus, retrovirus, and other delivery vehicles, such as, for example, liposomes and PLGA may be used to specifically target therapeutic moieties, such as, for example, IL-1 antagonist, GM-CSF antagonists, and the like, to particular cells. As is apparent to one skilled in the art, numerous other vaccines, drugs, and vectors may be useful in targeting and delivering therapeutic agents to particular cells.

Also provided herein are conjugates comprising a detectable (e.g., diagnostic) or therapeutic agent, attached to an antibody directed against a polypeptide translated from a nucleotide sequence chosen from SEQ ID NOs: 1-14 or their corresponding genes. Examples of such agents are well known, and include but are not limited to diagnostic radionuclides, therapeutic radionuclides, and cytotoxic drugs. See, e.g., Thrush et al., Annu. Rev. Immunol., 14:49-71, 1996. The conjugates may be useful in in vitro or in vivo procedures.

Fusion Proteins

Any polypeptide of the present invention can be used to generate fusion proteins. For example, the polypeptides of the present invention, when fused to a second protein, can be used as an antigenic tag. Antibodies raised against the polypeptides of the present invention can be used to indirectly detect the second protein by binding to the polypeptide. Moreover, because secreted proteins target cellular locations based on trafficking signals, the polypeptides of the present invention can be used as targeting molecules once fused to other proteins.

Examples of domains that can be fused to polypeptides of the present invention include not only heterologous signal sequences, but also other heterologous functional regions. The fusion does not necessarily need to be direct, but may occur through linker sequences.

Moreover, fusion proteins may also be engineered to improve characteristics of the polypeptide of the present invention. For instance, a region of additional amino acids, particularly charged amino acids, may be added to the N-terminus of the polypeptide to improve stability and persistence during purification from the host cell or subsequent handling and storage. Also, peptide moieties may be added to the polypeptide to facilitate purification. Such regions may be removed prior to final preparation of the polypeptide. The addition of peptide moieties to facilitate handling of polypeptides is a familiar and routine technique in the art.

In addition, polypeptides of the present invention, including fragments and, specifically, epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins (EP A 394,827; Traunecker et al., Nature, 331:84-86, 1988). Fusion proteins having disulfide-linked dimeric structures (due to the IgG) can also be more efficient in binding and neutralizing other molecules, than the monomeric secreted protein or protein fragment alone (Fountoulakis et al., J. Biochem., 270:3958-3964 (1995)).

Similarly, EP A 0 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties (see, e.g., EP A 0 232 262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5 (See, Bennett et al., J. Mol. Recognition 8:52-58 (1995); Johanson et al., J. Biol. Chem., 270:9459-9471, 1995).

Moreover, the polypeptides of the present invention can be fused to marker sequences, such as a peptide that facilitates purification of the fused polypeptide. In preferred embodiments, the marker amino acid sequence is a hexa-histidine peptide, such as the tag provided in a pQE vector (QIAGEN, Inc., Chatsworth, Calif.), among others, many of which are commercially available. As described in Gentz et al., for instance, hexa-histidine provides for convenient purification of the fusion protein (Proc. Natl. Acad. Sci. USA 86:821-824 (1989)). Another peptide tag useful for purification, the “HA” tag, corresponds to an epitope derived from the influenza hemagglutinin protein (Wilson et al., Cell, 37:767 (1984)). Other fusion proteins may use the ability of the polypeptides of the present invention to target the delivery of a biologically active peptide. This might include focused delivery of a toxin to tumor cells, or a growth factor to stem cells.

Thus, any of these above fusions can be engineered using the polynucleotides or the polypeptides of the present invention. See, e.g., Curr. Prot. Mol. Bio., Chapter 9.6.

Vectors, Host Cells, and Protein Production

The present invention also relates to vectors containing the polynucleotide or gene of the present invention or regions thereof, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

The polynucleotides, genes or regions thereof may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells. See, e.g., Curr. Prot. Mol Bio., Chapters 9.9, 16.15.

The polynucleotide or gene or gene region insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293, and Bowes melanoma cells, and plant cells. Appropriate culture mediums and conditions for the above-described host cells are known in the art.

Vectors preferred for use in bacteria include pQE70, pQE60 and pQE-9, available from QIAGEN, Inc.; pBluescript vectors, Phagescript vectors, pNH8A, PNH16A, PNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may, in fact, be expressed by a host cell lacking a recombinant vector.

A polypeptide of this invention can be recovered and purified from recombinant cell cultures by well-known methods, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Most preferably, high performance liquid chromatography (“HPLC”) is employed for purification.

Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

Polypeptides of the present invention, and preferably the secreted form, can also be recovered from products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells.

Other Uses of the Polynucleotides of the Invention

Each of the polynucleotides and genes of the present invention and regions thereof identified herein can be used in numerous ways as reagents. The following description should be considered exemplary and utilizes known techniques.

The polynucleotides and genes of the present invention and regions thereof are useful for chromosome identification. There exists an ongoing need to identify new chromosome markers, since few chromosome marking reagents based on actual sequence data (repeat polymorphisms) are presently available. Each polynucleotide of the present invention can be used as a chromosome marker. To date very few diagnostic markers exist for the detection of alcoholism. The present invention identifies genes that can be used as markers of alcoholism. For example, DST CAR1_{—}3 is a gene that may be differentially expressed as a result of 1 or 2 single nucleotide polymorphisms with decreased expression of both the mRNA and protein in the alcohol preferring rats. The single nucleotide polymorphisms represent diagnostic markers that can be used to identify this behavioral phenotype.

Briefly, sequences can be mapped to chromosomes by preparing PCR primers (preferably 15-25 bp) from the sequences shown in SEQ ID NOs:1-14 or their corresponding genes or regions thereof. Primers can be selected using computer analysis so that primers do not span more than one predicted exon in the genomic DNA. These primers may then be used for PCR screening of somatic cell hybrids containing individual human chromosomes. Only those hybrids containing the human gene corresponding to the SEQ ID NOs:1-14 or their corresponding genes or regions thereof will yield an amplified fragment.

Similarly, somatic hybrids provide a rapid method of PCR mapping the polynucleotides to particular chromosomes. Moreover, sublocalization of the polynucleotides can be achieved with panels of specific chromosome fragments. Other gene-mapping strategies that can be used include in situ hybridization, prescreening with labeled flow-sorted chromosomes, and preselection by hybridization to construct chromosome specific-cDNA libraries.

Precise chromosomal location of the polynucleotides, genes or regions thereof can also be achieved using fluorescence in situ hybridization (FISH) of a metaphase chromosomal spread. This technique uses polynucleotides as short as 500 or 600 bases; however, polynucleotides of 2,000-4,000 bp are preferred. For a review of this technique, see Verma et al., Human Chromosomes: a Manual of Basic Techniques, Pergamon Press, New York (1988).

For chromosome mapping, the polynucleotides, genes or regions thereof can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross-hybridization during chromosomal mapping.

Once a polynucleotide gene or region thereof has been mapped to a precise chromosomal location, the physical position of the polynucleotide gene or region thereof can be used in linkage analysis. Linkage analysis establishes coinheritance between a chromosomal location and presentation of a particular disease. Disease mapping data are found, for example, in V. McKusick, Mendelian Inheritance in Man (available on line through Johns Hopkins University Welch Medical Library), Kruglyak et al. (Am. J. Hum. Genet., 56:1212-23, 1995); Curr. Prot. Hum. Genet. Assuming one megabase mapping resolution and one gene per 20 kb, a cDNA precisely localized to a chromosomal region associated with the disease could be one of 50-500 potential causative genes. Thus, once coinheritance is established, differences in the polynucleotide and the corresponding gene or region thereof between affected and unaffected individuals can be examined.

The polynucleotides of SEQ ID NOs:1-14 and their corresponding genes or regions thereof can be used for individualized analysis. A genetic contribution to alcoholism is supported by adoption studies that demonstrate an increased risk for severe alcohol-related problems in children who were adopted out and had no knowledge of their biological parent's alcohol problems. Additionally, a number of studies have shown that alcoholism is greater than 50% heritable (Goldman, 1993; Reich et al., 1999). Therefore, the presence of a polynucleotide upregulated in alcohol seeking individuals versus non alcohol seeking individuals could be used to diagnose alcoholism.

For example, visible structural alterations in the chromosomes, such as deletions or translocations, are examined in chromosome spreads or by PCR. If no structural alterations exist, the presence of point mutations is ascertained. Mutations observed in some or all affected individuals, but not in normal individuals, indicates that the mutation may cause the disease. Such polymorphisms may be used to identify those individuals with a genetic susceptibility to alcoholism. For example, genetic tests designed to detect susceptibility alleles of genes such as DST CAR1_{—}3, may be used to predict which individuals are likely to develop alcoholism. However, complete sequencing of the polypeptide and the corresponding gene from several normal individuals is required to distinguish the mutation from a polymorphism. If a new polymorphism is identified, this polymorphic polypeptide can be used for further linkage analysis.

Furthermore, increased or decreased expression of the gene in affected individuals as compared to unaffected individuals can be assessed using polynucleotides or genes of the present invention or regions thereof. Any of these alterations (altered expression, chromosomal rearrangement, or mutation) can be used as a diagnostic or prognostic marker.

In addition to the foregoing, a polynucleotide or gene of the invention or regions thereof can be used to control gene expression through triple helix formation or antisense DNA or RNA. Both methods rely on binding of the polynucleotide or gene or gene region to DNA or RNA. For these techniques, preferred polynucleotides are usually 20 to 40 bases in length and complementary to either the region of the gene involved in transcription (see, Lee et al., Nuc. Acids Res., 6:3073 (1979); Cooney et al., Science, 241:456 (1988); and Dervan et al., Science, 251:1360 (1991) for discussion of triple helix formation) or to the mRNA itself (see, Okano, J. Neurochem, 56:560 (1991); and Oligodeoxy-nucleotides as Antisense Inhibitors of Gene Expression, CRC Press, Boca Raton, Fla. (1988) for a discussion of antisense technique). Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat disease.

Other Uses of the Polypeptides and Antibodies of the Invention

Each of the polypeptides identified herein can be used in numerous ways. The following description should be considered exemplary and utilizes known techniques.

A polypeptide of the present invention can be used to assay protein levels in a biological sample using antibody-based techniques. For example, protein expression in tissues can be studied with classical immunohistological methods (Jalkanen, et al., J. Cell. Biol., 101:976-985, 1985; Jalkanen et al., J. Cell. Biol., 105:3087-3096, 1987). Other antibody-based methods useful for detecting protein gene expression include immunoassays, such as the enzyme linked immunosorbent assay (ELISA) and the radioimmunoassay (RIA). See, e.g., Curr. Prot. Mol. Bio., Chapter 11. Suitable antibody assay labels are known in the art and include enzyme labels, such as glucose oxidase; and radioisotopes, such as iodine (¹²⁵I, ¹²¹I), carbon (¹⁴C), sulfur (³⁵S), tritium (³H), indium (¹¹²In), and technetium (^{99m}Tc); fluorescent labels, such as fluorescein and rhodamine; and organic moieties, such as biotin.

In addition to assaying secreted protein levels in a biological sample, proteins can also be detected in vivo by imaging. Antibody labels or markers for in vivo imaging of protein include those detectable by X-radiography, nuclear magnetic resonance (NMR), or electron spin resonance (ESR). For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment that has been labeled with an appropriate detectable imaging moiety such as a radioisotope (e.g., ¹³¹I, ¹¹²In, ^{99m}Tc), a radio-opaque substance, or a material detectable by NMR, is introduced (e.g., parenterally, subcutaneously, or intraperitoneally) into the mammal. It will be understood in the art that the size of the subject and the imaging system used will determine the quantity of imaging moiety needed to produce diagnostic images. In the case of a radioisotope moiety, the quantity of radioactivity necessary for a human subject will normally range from about 5 to 20 millicuries of ^{99m}Tc. The labeled antibody or antibody fragment will then preferentially accumulate at the location of cells which contain the specific protein. In vivo tumor imaging is described in Burchiel et al., “Immunopharmacokinetics of Radiolabeled Antibodies and Their Fragments” (Chapter 13 in Tumor Imaging: The Radiochemical Detection of Cancer, Burchiel and Rhodes, Eds., Masson Publishing Inc. (1982)).

Thus, the invention provides a method of diagnosing alcoholism, which involves (a) assaying the expression of a polypeptide of the present invention in cells or body fluid of an individual; and (b) comparing the level of gene expression with a standard gene expression level, whereby an increase or decrease in the assayed polypeptide gene expression level compared to the standard expression level is indicative of alcoholism.

Moreover, polypeptides of the present invention can be used to treat alcoholism. For example, patients can be administered a polypeptide of the present invention in an effort to replace absent or decreased levels of the polypeptide (e.g., insulin); to supplement absent or decreased levels of a different polypeptide (e.g., hemoglobin S for hemoglobin B); to inhibit the activity of a polypeptide (e.g., an oncogene); to activate the activity of a polypeptide (e.g., by binding to a receptor); to reduce the activity of a membrane bound receptor by competing with it for free ligand (e.g., soluble tumor necrosis factor (TNF) receptors used in reducing inflammation); or to bring about a desired response (e.g., blood vessel growth).

Similarly, antibodies directed to a polypeptide of the present invention can also be used to treat alcoholism. For example, administration of an antibody directed to a polypeptide of the present invention can bind and reduce overproduction of the polypeptide. Similarly, administration of an antibody can activate the polypeptide, such as by binding to a polypeptide bound to a membrane (receptor). Polypeptides can be used as antigens to trigger immune responses.

A mammalian subject (preferably a human) can be given a recombinant or synthetic form of a polypeptide or antibody in one of many possible different formulations, preferably encapsulated and other forms for oral or other gastrointestinal delivery of the polypeptide or antibody. In some cases, delivery of the polypeptide or antibody may be in the form of injection or transplantation of cells or tissues containing an expression vector such that a recombinant form of the polypeptide will be secreted by the cells or tissues, as described above for transfected cells.

The frequency and dosage of the administration of the polypeptides or antibodies will be determined by factors such as the biological activity of the pharmacological preparation and the goals to decrease the desire to consume alcohol. In the case of antibody deliveries, the frequency of dosage will also depend on the ability of the antibody to bind and neutralize the target molecules in the target tissues.

Polypeptides can also be used to raise antibodies, which in turn are used to measure protein expression from a recombinant cell, as a way of assessing transformation of the host cell. See, e.g., Curr. Prot. Mol. Bio., Chapter 11.15. Moreover, the polypeptides of the present invention can be used to test the following biological activities.

Biological Activities

The polynucleotides, polypeptides and genes of the present invention and regions thereof can be used in assays to test for one or more biological activities. If these polynucleotides, polypeptides and genes or gene regions exhibit activity in a particular assay, it is likely that these molecules may be involved in the diseases associated with biological activity.

Binding Activity

A polypeptide of the present invention may be used to screen for molecules that bind to the polypeptide or for molecules to which the polypeptide binds. The binding of the polypeptide and the molecule may activate (i.e., an agonist), increase, inhibit (i.e., an antagonist), or decrease activity of the polypeptide or the molecule bound. Examples of such molecules include antibodies, oligonucleotides, proteins (e.g., receptors), or small molecules.

Preferably, the molecule is closely related to the natural ligand of the polypeptide, e.g., a fragment of the ligand, or a natural substrate, a ligand, a structural or functional mimetic (see, e.g., Coligan et al., Current Protocols in Immunology 1(2), Chapter 5 (1991)). Similarly, the molecule can be closely related to the natural receptor to which the polypeptide binds or, at least, related to a fragment of the receptor capable of being bound by the polypeptide (e.g., an active site). In either case, the molecule can be rationally designed using known techniques.

Preferably, the screening for these molecules involves producing appropriate cells which express the polypeptide, either as a secreted protein or on the cell membrane. Preferred cells include cells from mammals, yeast, Drosophila, or E. coli. Cells expressing the polypeptide (or cell membrane containing the expressed polypeptide) are then preferably contacted with a test compound potentially containing the molecule to observe binding, stimulation, or inhibition of activity of either the polypeptide or the molecule.

The assay may simply test binding of a candidate compound to the polypeptide, wherein binding is detected by a label, or in an assay involving competition with a labeled competitor. Further, the assay may test whether the candidate compound results in a signal generated by binding to the polypeptide.

Alternatively, the assay can be carried out using cell-free preparations, polypeptide/molecule affixed to a solid support, chemical libraries, or natural product mixtures. The assay may also simply comprise the steps of mixing a candidate compound with a solution containing a polypeptide, measuring polypeptide/molecule activity or binding, and comparing the polypeptide/molecule activity or binding to a standard.

Preferably, an ELISA assay can measure polypeptide level or activity in a sample (e.g., biological sample) using a monoclonal or polyclonal antibody. The antibody can measure polypeptide level or activity by either binding, directly or indirectly, to the polypeptide or by competing with the polypeptide for a substrate.

All of these above assays can be used as diagnostic or prognostic markers. The molecules discovered using these assays can be used to treat disease or to bring about a particular result in a patient (e.g., blood vessel growth) by activating or inhibiting the polypeptide/molecule. Moreover, the assays can discover agents that may inhibit or enhance the production of the polypeptide from suitably manipulated cells or tissues. Diagnosis depends on a number of relatively invasive and expensive clinical tests. Assays for the presence of markers, in easily obtained specimens (blood, urine or stool) may provide an important diagnostic tool.

Therefore, the invention includes a method of identifying compounds which bind to a polypeptide of the invention comprising the steps of: (a) incubating a candidate binding compound with a polypeptide of the invention; and (b) determining if binding has occurred. Moreover, the invention includes a method of identifying agonists/antagonists comprising the steps of: (a) incubating a candidate compound with a polypeptide of the invention, (b) assaying a biological activity, and (c) determining if a biological activity of the polypeptide has been altered.

Other Activities

A polypeptide, polynucleotide or gene of the present invention or a region thereof may also increase or decrease the differentiation or proliferation of embryonic stem cells from a lineage other than the above-described hemopoietic lineage. Alcoholism has been demonstrated to result in the death of cells in the brain. A polypeptide, polynucleotide or gene of the present invention or region thereof may be used to modulate the development and differentiation of nervous system precursors to be used to generate and promote the growth of cells to replace those lost.

A polypeptide, polynucleotide or gene of the present invention or a region thereof may also be used to modulate mammalian characteristics, such as body height, weight, hair color, eye color, skin, percentage of adipose tissue, pigmentation, size, and shape (e.g., cosmetic surgery). Similarly, a polypeptide, polynucleotide, or gene of the present invention or region thereof may be used to modulate mammalian metabolism affecting catabolism, anabolism, processing, utilization, and storage of energy.

A polypeptide, polynucleotide or gene of the present invention or a region thereof may be used to change a mammal's mental state or physical state by influencing biorhythms, circadian rhythms, depression (including depressive disorders), tendency for violence, tolerance for pain, the response to opiates and opioids, tolerance to opiates and opioids, withdrawal from opiates and opioids, reproductive capabilities (preferably by activin or inhibin-like activity), hormonal or endocrine levels, appetite, libido, memory, stress, or other cognitive qualities.

A polypeptide, polynucleotide or gene of the present invention or a region thereof may also be used as a food additive or preservative, such as to increase or decrease storage capabilities, fat content, lipid, protein, carbohydrate, vitamins, minerals, cofactors, or other nutritional components.

The following examples are intended to illustrate the invention, and are not to be construed as limiting the scope of the invention.

### EXAMPLES

### Example 1

### Identification of Genes Regulated in Alcoholism

To identify genes that are differentially expressed in alcohol-preferring as compared to alcohol-non-preferring rats, mRNA from 4 specific brain regions was analyzed using TOGA®. Once the regulated genes were identified, several analyses were conducted. The regulated gene products from the alcohol-preferring and alcohol-non-preferring rats were sequenced and compared to identify polymorphisms that might contribute the to differential gene expression. The regulated DSTs were localized to specific brain regions by in situ hybridization. The DSTs were mapped to determine whether they localize to a specific QTL associated with alcohol seeking behavior. The protein expression levels of the regulated DSTs were determined to verify that the difference in gene expression corresponded to a difference in protein expression.

### Example 2

### Generation of Alcohol Inbred Preferring and Alcohol Inbred Non-Preferring Rats

Alcohol inbred preferring and alcohol inbred non-preferring rats were generated by selectively breeding animals for a particular phenotypic trait and then, in some cases, completing many generations of brother-sister mating to produce highly inbred strains with the phenotype of interest. The alcohol-preferring (P) and alcohol-non-preferring (NP) rat lines were developed at Indiana University for high and low alcohol preference behavior through bidirectional selective breeding from a randomly bred closed colony of Wistar rats (Wrm: WRC(WI)BR) from the Walter Reed Army Institute of Research, Washington, D.C. (Li et al, 1991). Following successful divergence and plateau of the phenotype in each of the selected lines, brother-sister mating was initiated at generation 30 of selection to develop inbred lines. At 31 generations of inbreeding, 10 alcohol-naïve inbred male P rats and 10 alcohol-naïve inbred male NP rats that were three months of age were used in the present invention.

### Example 3

### Micro-Dissection-and Isolation of Specific Brain Regions

Entire brains were removed from sacrificed rats. The brain tissue was micro-dissected to obtain 4 sub-region samples: 1) hypothalamus, 2) hippocampus, 3) caudate-putamen, nucleus accumbens and olfactory tubercles, and 4) prefrontal-, frontal- and parietal-cortex. Micro-dissection was conducted as follows: 1) the hypothalamus was removed by pinching off the sub-region (measuring approximately 4 mm×4 mm×2 mm) from the ventral (base) surface of the brain using a pair of curved tweezers; 2) the olfactory tubercles were removed from the very rostral extent of the brain by pinching off the tissue (measuring approximately 2 mm×2 mm×3 mm) with forceps proximal to the attachment point of the olfactory bulbs; 3) the hippocampus was removed by rolling the cortical hemispheres forward from the cerebellar attachment site, and then gently teasing the CA1, CA2 and CA3 regions from the base of the exposed cortex under-layer; 4) the caudate-putamen and nucleus accumbens was removed by: cutting the brain in half down the center line (rostral-to-caudal); teasing forward the most rostral portion of the cortex and pinching-off a 3 mm×3 mm×3 mm plug of tissue bounded by the presence of dense white-matter tracts; 5) the prefrontal-, frontal- and parietal-cortex sample was prepared by cutting through the remaining tissue in a rostral-to-caudal direction dorsal to the removed striatum and along a curve parallel to the surface of the cerebral cortex. RNA was isolated according to standard methods.

### Example 4

### The TOGA® Process

Isolated RNA was analyzed using TOGA®. Preferably, prior to TOGA®, the isolated RNA was enriched to form a starting polyA-containing mRNA population by methods known in the art. In a preferred embodiment, the TOGA® method further comprised an additional PCR step performed using four 5′ PCR primers in four separate reactions and cDNA templates prepared from a population of antisense cRNAs. A final PCR step that used 256 5′ PCR primers in separate reactions produced PCR products that were cDNA fragments that corresponded to the 3′-region of the starting mRNA population. The produced PCR products were then identified by: a) the initial 5′ sequence comprising the sequence remainder of the recognition site of the restriction endonuclease used to cut and isolate the 3′ region plus the sequence of the preferably four parsing bases immediately 3′ to the remainder of the recognition site, preferably the sequence of the entire fragment, and b) the length of the fragment. These two parameters, sequence and fragment length, were used to compare the obtained PCR products to a database of known polynucleotide sequences. Since the length of the obtained PCR products includes known vector sequences at the 5′ and 3′ ends of the insert, the sequence of the insert provided in the sequence listing is shorter than the fragment length that forms part of the digital address.

The method yields Digital Sequence Tags (DSTs), that is, polynucleotides that are expressed sequence tags of the 3′ end of mRNAs. DSTs that showed changes in relative levels in the alcohol-preferring rat brain regions versus the alcohol-non-preferring rat brain regions were selected for further study. The intensities of the laser-induced fluorescence of the labeled PCR products were compared across sample isolated from the alcohol-preferring rats and the alcohol-non-preferring rats.

For the hippocampus, cortex, and hypothalamus samples, double-stranded cDNA was generated from poly(A)-enriched cytoplasmic RNA extracted from the tissue samples of interest using an equimolar mixture or set of all 48 5′-biotinylated anchor primers to initiate reverse transcription. One such suitable set is G-A-A-T-T-C-A-A-C-T-G-G-A-A-G-C-G-G-C-C-G-C-A-G-G-A-A-T-T-T-T-T-T-T-T-T-T-T-T-T-T-T-T-T-T-V-N-N (SEQ ID NO: 14), where V is A, C or G and N is A, C, G or T. One member of this mixture of 48 anchor primers initiates synthesis at a fixed position at the 3′ end of all copies of each mRNA species in the sample, thereby defining a 3′ endpoint for each species, resulting in biotinylated double-stranded cDNA.

For the striatum samples, double-stranded cDNA is generated from poly(A)-enriched cytoplasmic RNA extracted from the tissue samples of interest using an equimolar mixture or set of all 48 5′-biotinylated anchor primers to initiate reverse transcription. One such suitable set is G-A-A-T-T-C-A-A-C-T-G-G-A-A-G-C-G-G-C-C-G-C-A-G-G-A-A-G-A-G-C-T-C-C-A-C-C-G-C-G-G-T-A-G-T-A-C-T-C-A-C-T-G-C-A-G-T-T-T-T-T-T-T-T-T-T-T-T-T-T-T-T-T-T-V-N-N (SEQ ID NO: 19), where V is A, C or G and N is A, C, G or T. One member of this mixture of 48 anchor primers initiates synthesis at a fixed position at the 3′ end of all copies of each mRNA species in the sample, thereby defining a 3′ endpoint for each species, resulting in biotinylated double-stranded cDNA.

Each biotinylated double-stranded cDNA sample was cleaved with the restriction endonuclease MspI, which recognizes the sequence CCGG. The resulting fragments of cDNA corresponding to the 3′ region of the starting mRNA were then isolated by capture of the biotinylated cDNA fragments on a streptavidin-coated substrate. Suitable streptavidin-coated substrates include microtitre plates, PCR tubes, polystyrene beads, paramagnetic polymer beads, and paramagnetic porous glass particles. A preferred streptavidin-coated substrate is a suspension of paramagnetic polymer beads (Dynal, Inc., Great Neck, N.Y.).

After washing the streptavidin-coated substrate and captured biotinylated cDNA fragments, the cDNA fragment product was released by digestion with NotI, which cleaves at an 8-nucleotide sequence within the anchor primers but rarely within the mRNA-derived portion of the cDNAs. The 3′ MspI-NotI fragments, which are of uniform length for each mRNA species, were directionally ligated into ClaI-NotI-cleaved plasmid pBC SK+ (Stratagene, La Jolla, Calif.) in an antisense orientation with respect to the vector's T3 promoter, and the product used to transform Escherichia coli SURE cells (Stratagene). The ligation regenerates the NotI site, but not the MspI site, leaving CGG as the first 3 bases of the 5′ end of all PCR products obtained. Each library contained in excess of 5×10⁵ recombinants to ensure a high likelihood that the 3′ ends of all mRNAs with concentrations of 0.001% or greater were multiply represented. Plasmid preps (Qiagen) were made from the cDNA library of each sample under study.

An aliquot of each library was digested with MspI, which effects linearization by cleavage at several sites within the parent vector while leaving the 3′ cDNA inserts and their flanking sequences, including the T3 promoter, intact. The product was incubated with T3 RNA polymerase (MEGASCRIPT kit, Ambion) to generate antisense cRNA transcripts of the cloned inserts containing known vector sequences abutting the MspI and NotI sites from the original cDNAs.

At this stage, each of the cRNA preparations was processed in a three-step fashion. In step one, 250 ng of cRNA was converted to first-strand cDNA using the 5′ RT primer (A-G-G-T-C-G-A-C-G-G-T-A-T-C-G-G, (SEQ ID NO: 15). In step two, 400 pg of cDNA product was used as PCR template in four separate reactions with each of the four 5′ PCR primers of the form G-G-T-C-G-A-C-G-G-T-A-T-C-G-G-N (SEQ ID NO: 16), each paired with a “universal” 3′ PCR primer G-A-G-C-T-C-C-A-C-C-G-C-G-G-T (SEQ ID NO: 17) to yield four sets of PCR reaction products (“N1 reaction products”).

In step three, the product of each subpool was further divided into 64 subsubpools (2 ng in 20 μl) for the second PCR reaction. This PCR reaction comprised adding 100 ng of the fluoresceinated “universal” 3′ PCR primer (SEQ ID NO: 17) conjugated to 6-FAM and 100 ng of the appropriate 5′ PCR primer of the form C-G-A-C-G-G-T-A-T-C-G-G-N-N-N-N (SEQ ID NO: 18), and using a program that included an annealing step at a temperature X slightly above the Tm of each 5′ PCR primer to minimize artifactual mispriming and promote high fidelity copying. Each polymerase chain reaction step was performed in the presence of TaqStart antibody (Clonetech).

Each biotinylated double-stranded cDNA sample was cleaved with the restriction endonuclease MspI, which recognizes the sequence CCGG. The resulting fragments of cDNA corresponding to the 3′ region of the starting mRNA were then isolated by capture of the biotinylated cDNA fragments on a streptavidin-coated substrate. Suitable streptavidin-coated substrates include microtitre plates, PCR tubes, polystyrene beads, paramagnetic polymer beads, and paramagnetic porous glass particles. A preferred streptavidin-coated substrate is a suspension of paramagnetic polymer beads (Dynal, Inc., Great Neck, N.Y.).

The pool or library of captured cDNA products was modified by ligation of double-stranded polynucleotides at the 5′ ends to contain sequences encoding a T3 RNA polymerase promoter and PCR primer binding sites. If the biotinylated cDNA samples were processed with the restriction enzyme MspI, suitable polynucleotides are A-A-T-T-C-G-G-T-A-C-C-A-A-T-T-A-A-C-C-C-T-C-A-C-T-A-A-A-G-G-G-A-C-C-T-C-G-A-G-G-T-C-G-A-C-G-G-T-A-T and C-G-A-T-A-C-C-G-T-C-G-A-C-C-T-C-G-A-G-G-T-C-C-C-T-T-T-A-G-T-G-A-G-G-G-T-T-A-A-T-T-G-G-T-A-C-C-G-A-A-T-T (SEQ ID NOs:20 and 21, respectively). The modified cDNA library was subsequently used as a template for synthesis of cRNA (copy RNA) by incubation with T3 RNA polymerase.

At this stage, each of the cRNA preparations was processed in a three-step fashion. In step one, an aliquot of cRNA was used for synthesis of first-strand cDNA using the 5′ RT primer (G-A-G-C-T-C-C-A-C-C-G-C-G-G-T, (SEQ ID NO:22). In step two, the cDNA product was used as a DNA template in four separate PCR reactions with each of the four 5′ PCR primers of the form C-C-T-C-G-A-G-G-T-C-G-A-C-G-G-T-A-T-C-G-G-N (SEQ ID NO:23), each paired with a “universal” 3′ PCR primer G-A-G-C-T-C-C-A-C-C-G-C-G-G-T (SEQ ID NO: 17) to yield four sets of PCR reaction products (“N1 reaction products”). In step three, the product of each subpool was further divided into 64 subsubpools (2 ng in 20 μl) for the second PCR reaction. This PCR reaction comprised adding 100 ng of the fluoresceinated “universal” 3′ PCR primer (SEQ ID NO: 22) conjugated to 6-FAM and 100 ng of the appropriate 5′ PCR primer of the form C-G-A-C-G-G-T-A-T-C-G-G-N-N-N-N (SEQ ID NO: 18) and using a program that included an annealing step at a temperature X slightly above the Tm of each 5′ PCR primer to minimize artifactual mispriming and promote high fidelity copying. Each polymerase chain reaction step was performed in the presence of TaqStart antibody (Clonetech).

The products (“N4 reaction products”) from the final polymerase chain reaction step for each of the samples were resolved on a series of denaturing DNA sequencing gels using the automated ABI Prizm 377 sequencer. Data were collected using the GENESCAN software package (ABI) and normalized for amplitude and migration. In some experiments, the products were resolved by capillary electrophoresis on MEGABACE 1000 instruments (Amersham). Data from the MEGABACE 1000 were collected and processed with custom software (Digital Gene Technologies), which included size calibration and amplitude and migration normalization. Complete execution of this series of reactions generated 64 product subpools for each of the four pools established by the 5′ PCR primers of the first PCR reaction, for a total of 256 product subpools for the entire 5′ PCR primer set of the second PCR reaction.

The mRNA samples from alcohol-preferring and alcohol-non-preferring rat brains were analyzed. Table 1 summarizes the expression levels of 7 mRNAs determined from cDNA. These cDNA molecules are identified by their digital address, that is, a partial 5′ terminus nucleotide sequence coupled with the length of the molecule, as well as the relative amount of the molecule produced at different time intervals after treatment. The 5′ terminus partial nucleotide sequence is determined by the recognition site for MspI (CCGG) and the nucleotide sequence of the parsing bases of the 5′ PCR primer used in the final PCR step. The digital address length of the fragment was determined by interpolation on a standard curve and, as such, may vary +1-2 b.p. from the actual length.

For example, the entry in Table 1 that describes a DNA molecule identified by the digital address MspI ATGG266, is further characterized as having a 5′ terminus partial nucleotide sequence of CGGATGG and a length of 266 b.p. The DNA molecule identified as MspI ATGG266 is further described as being expressed rat hippocampus. Additionally, the DNA molecule identified as MspI ATGG266 is described by its nucleotide sequence, which corresponds with SEQ ID NO: 1.

Similarly, the other DNA molecules identified in Table 1 by their MspI digital addresses are further characterized by: the level of gene expression in the alcohol-preferring rat brain regions as compared to the alcohol-non-preferring rat brain regions.

Additionally, several of the DSTs were further characterized in the Tables and Sequence Listing below.

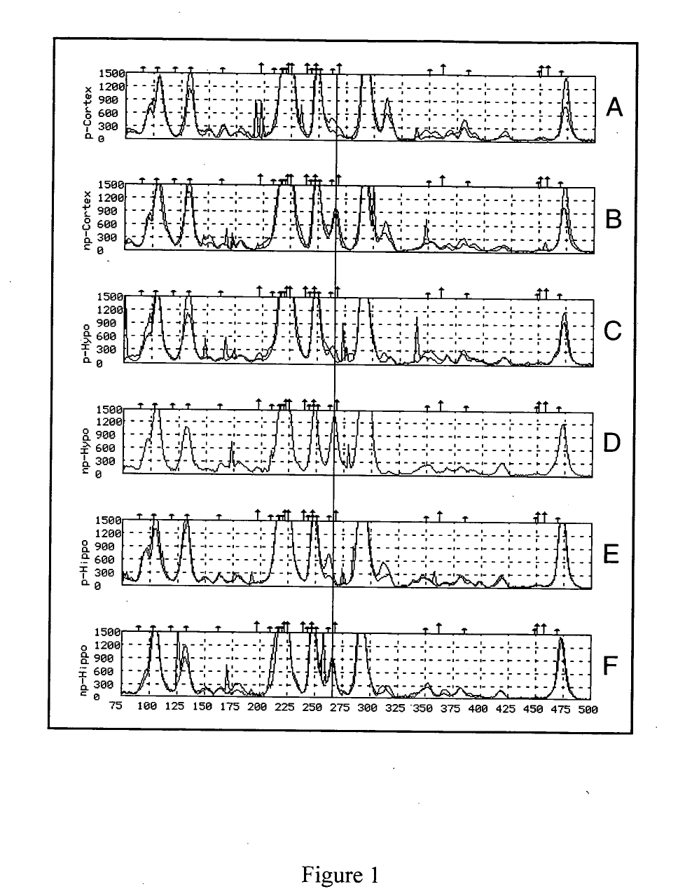

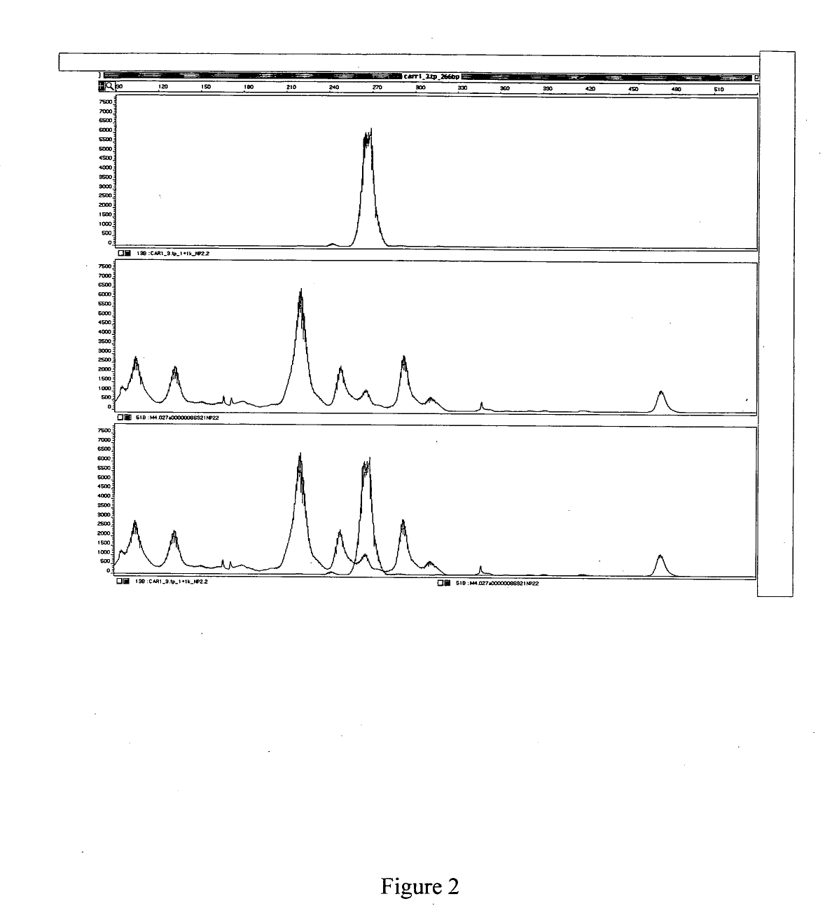

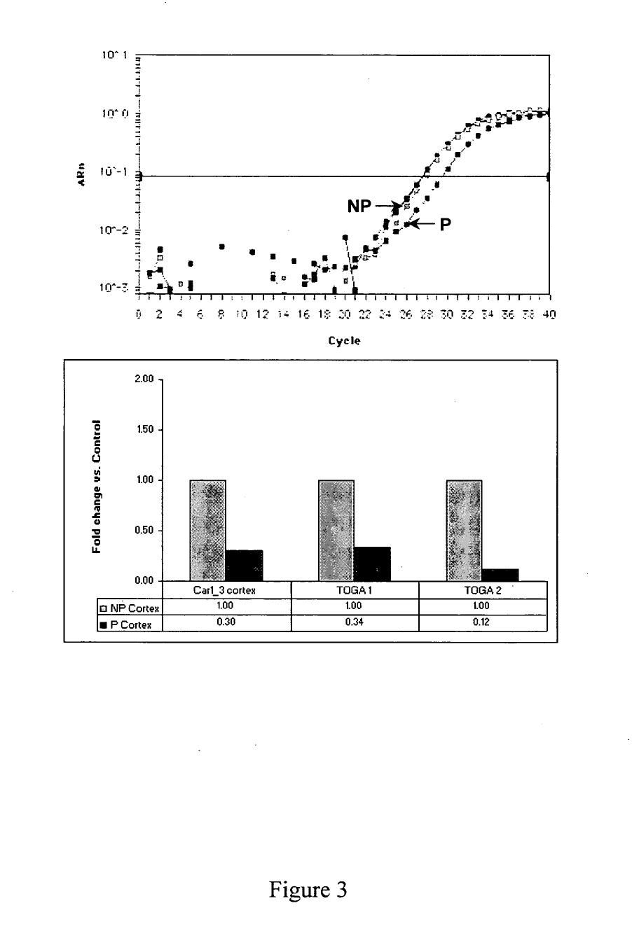

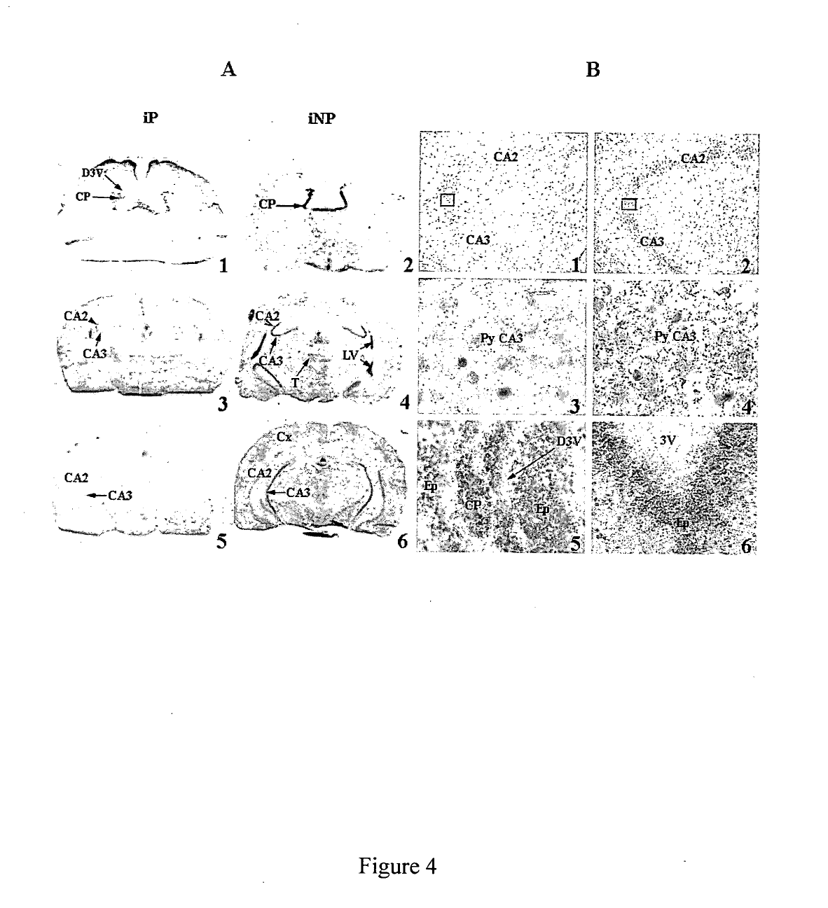

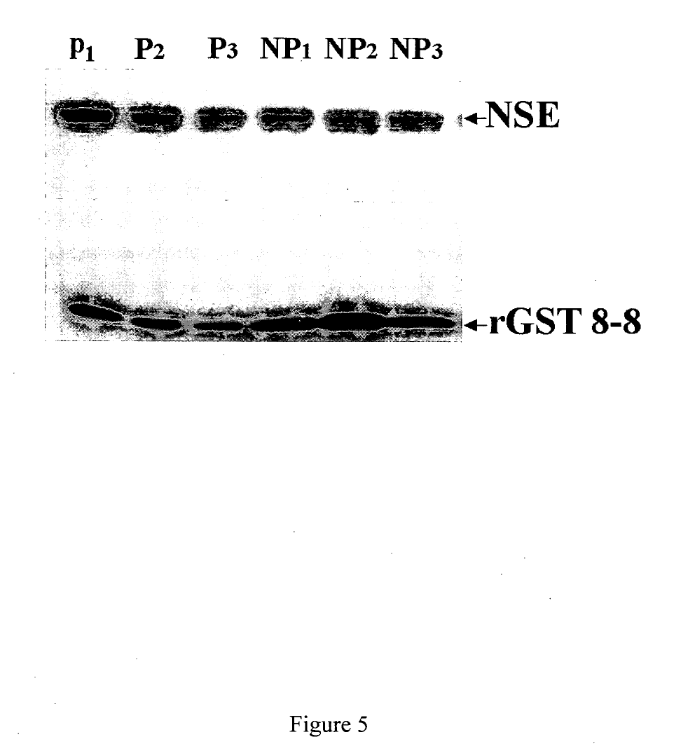

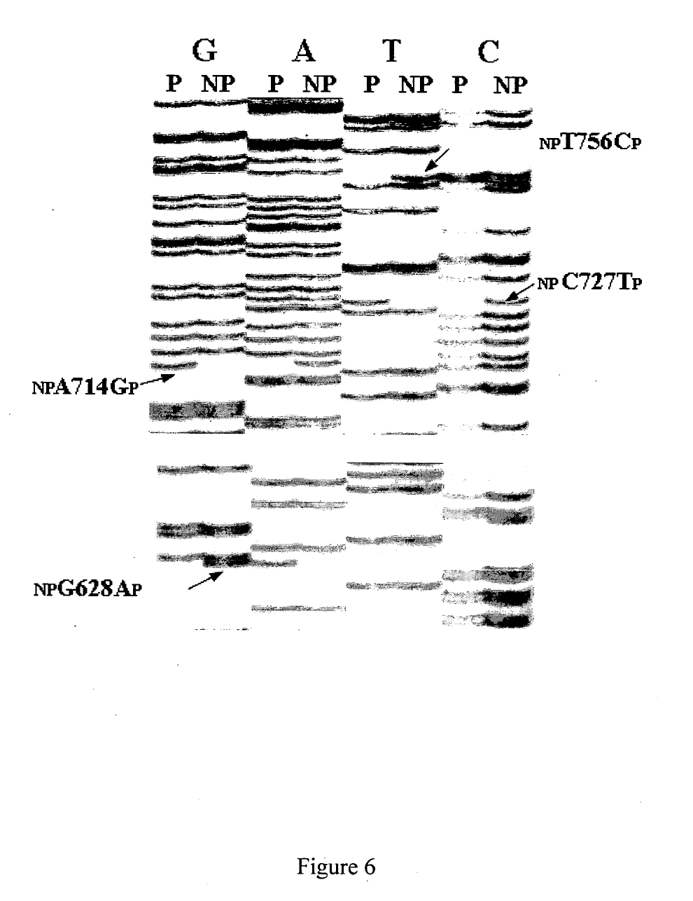

The data shown in FIG. 1 were generated with a 5′-PCR primer (C-G-A-C-G-G-T-A-T-C-G-G-A-T-G-G (SEQ ID NO: 24) paired with the “universal” 3′ primer (SEQ ID NO:17) labeled with 6-carboxyfluorescein (6FAM, ABI) at the 5′ terminus. PCR reaction products were resolved by gel electrophoresis on 4.5% acrylamide gels and fluorescence data acquired on ABI377 automated sequencers. Data were analyzed using GENESCAN software (Perkin-Elmer). The sequences of the PCR products were determined using standard techniques.

FIG. 1 is a graphical representation of the results of TOGA® analysis using a 5′ PCR primer with parsing bases ATGG (SEQ ID NO: 26) and the universal 3′ PCR primer (SEQ ID NO:17), which shows the PCR products produced from mRNA extracted from the cortex of the P rat brain (Panel A), the cortex of the NP rat brain (Panel B), the hypothalamus of the P rat brain (Panel C), and the hypothalamus of the NP rat brain (Panel D), the hippocampus of the P rat brain (Panel E), and the hippocampus of the NP rat brain (Panel F), where the vertical index line indicates a PCR product of about 266 b.p. The PCR product corresponds to an mRNA in the sample whose expression increases in the P rat hippocampus. The horizontal axis represents the number of base pairs of the PCR product and the vertical axis represents the fluorescence measurement in the TOGA® analysis corresponding to the relative expression of the molecule in the sample.

The results of the TOGA® runs were normalized as described above. The vertical line drawn through the 6 panels indicates the location of the molecule identified as CAR1_{—}3 (SEQ ID NO:1).

Some products, which were also differentially represented, appeared to migrate in positions that suggest that the products were novel based on comparison to data extracted from GenBank. The sequences of such products were determined by one of three methods: cloning, direct sequencing of the PCR products, or candidate matches with existing databases.

Cloning of TOGA® Generated PCR Products

In suitable cases, the PCR product was isolated, cloned into a TOPO vector (Invitrogen) and sequenced. Table 2 contains the database matches for the sequences determined by this method or by direct sequencing. In order to verify that the cloned product corresponds to the TOGA® peak of interest, the extended TOGA® assay was performed for each DST (see below).

Direct Sequencing of TOGA® Generated PCR Products

In other cases, the TOGA® PCR product was sequenced using a modification of a direct sequencing methodology (Innis et al., Proc. Nat'l. Acad. Sci., 85: 9436-9440, 1988).

PCR products corresponding to DSTs were gel purified and PCR amplified again to incorporate sequencing primers at the 5′- and 3′-ends. The sequence addition was accomplished through 5′ and 3′ ds-primers containing M13 sequencing primer sequences (Ml 3 forward and M13 reverse respectively) at their 5′ ends, followed by a linker sequence and a sequence complementary to the DST ends. Using the Clontech Taq Start antibody system, a master mix containing all components except the gel purified PCR product template was prepared, which contained sterile H₂O, 10×PCR II buffer, 10 mM dNTP, 25 mM MgCl₂, AmpliTaq/Antibody mix (1.1 μg/μl Taq antibody, 5 U/ml AmpliTaq), 100 ng/μl of 5′ ds-primer (5′ TCC CAG TCA CGA CGT TGT AAA ACG ACG GCT CAT ATG AAT TAG GTG ACC GAC GGT ATC GG 3′, SEQ ID NO:28), and 100 ng/μl of 3′ ds-primer (5′ CAG CGG ATA ACA ATT TCA CAC AGG GAG CTC CAC CGC GGT GGC GGC C₃′, SEQ ID NO:29). After addition of the PCR product template, PCR was performed using the following program: 94° C., 4 minutes and 25 cycles of 94° C., 20 seconds; 65° C., 20 seconds; 72° C., 20 seconds; and 72° C. 4 minutes. The resulting amplified PCR product was gel purified.

The purified PCR product was sequenced using a standard protocol for ABI 3700 sequencing. Briefly, triplicate reactions in forward and reverse orientation (6 total reactions) were prepared, each reaction containing 5 μg of gel purified PCR product as template. In addition, the sequencing reactions contained 2 μl 2.5× sequencing buffer, 2 μl Big Dye Terminator mix, 1 μl of either the 5′ sequencing primer (5′ CCC AGT CAC GAC GTT GTA AAA CG 3′, SEQ ID NO:28), or the 3′ sequencing primer (5′ TTT TTT TTT TTT TTT TTT V 3′, where V=A, C, or G, SEQ ID NO:29) in a total volume of 10 μl.

In an alternate embodiment, the 3′ sequencing primer was the sequence 5′ GGT GGC GGC CGC AGG AAT TTT TTT TTT TTT TTT TT 3′, (SEQ ID NO:30). PCR was performed using the following thermal cycling program: 96° C., 2 minutes and 29 cycles of 96° C., 15 seconds; 50° C., 15 seconds; 60° C., 4 minutes.

Table 2 lists the database matches for the sequences determined by this method or by cloning.

Verification Using the Extended TOGA® Method

In order to verify that the TOGA® peak of interest corresponds to the identified DST, an extended TOGA® assay was performed for each DST as described below. PCR primers (“Extended TOGA® primers”) were designed from a sequence determined using one of three methods: (1) in suitable cases, the PCR product was isolated, cloned into a TOPO vector (Invitrogen) and sequenced on both strands; or (2) in other cases, the TOGA® PCR product was sequenced using a modification of a direct sequencing methodology (Innis, Proc Natl Acad Sci, 1988), or (3) in other cases, the sequences listed for the TOGA® PCR products were derived from candidate matches to sequences present in available GenBank, EST, or proprietary databases.

PCR was performed using the Extended TOGA® primers and the N1 PCR reaction products as a substrate. Oligonucleotides were synthesized with the sequence G-A-T-C-G-A-A-T-C extended at the 3′ end with a partial MspI site (C-G-G), and an additional 18 adjacent nucleotides from the determined sequence of the DST. For example, for the PCR product with the TOGA® address ATGG266 (DST CAR1_{—}3); (SEQ ID NO:1), the 5′ PCR primer was G-A-T-C-G-A-A-T-C-C-G-G-T-G-C-T-C-C-C-C-T-C-T-C-A-C-T-A-C-A (SEQ ID NO:25). This 5′ PCR primer was paired with the fluorescence labeled universal 3′ PCR primer (SEQ ID NO:17) in a PCR reaction using the PCR N1 reaction product as substrate.

The length of the PCR product generated with the Extended TOGA® primer was compared to the length of the original PCR product that was produced in the TOGA® reaction. The results for SEQ ID NO:1, for example, are shown in FIG. 2. The length of the PCR product corresponding to SEQ ID NO:1 (DST CAR1_{—}3) was cloned and a 5′ PCR primer was built from the determined sequence (SEQ ID NO:25). The product obtained from PCR with this primer (SEQ ID NO:25) and the universal 3′ PCR primer (SEQ ID NO:17) (as shown in the top panel) was compared to the length of the original PCR product that was produced in the TOGA® reaction with mRNA extracted from the alcohol-non-preferring cortex sample using a 5′ PCR primer with parsing bases ATGG (SEQ ID NO:24) and the universal 3′ PCR primer (SEQ ID NO:17) (as shown in the middle panel). Again, for all panels, the number of base pairs is shown on the horizontal axis, and fluorescence intensity (which corresponds to relative expression) is found on the vertical axis. In the bottom panel, the traces from the top and middle panels are overlaid, demonstrating that the peak found using an extended primer from the cloned DST is the same number of base pairs as the original PCR product obtained through TOGA® as DST CAR1_{—}3 (SEQ ID NO:1).

In other cases, the sequences listed for the TOGA® PCR products were derived from candidate matches to sequences present in available GenBank, EST, or proprietary databases. Table 3 lists the candidate matches for each by accession number of the GenBank entry. Table 3 lists the database matches for DST sequences that were determined from proprietary databases or by the creation of consensus sequences based on sets of computer-assembled ESTs. Extended TOGA® primers were designed based on these sequences (as mentioned previously), and Extended TOGA® was run to determine if the database sequences were the DSTs amplified in TOGA®.

### Example 5

### Assignment of Identities to DSTs

Digital Sequence Tags (DSTs) can be associated with the gene encoding the full-length mRNA transcript including both 5′ and 3′ untranslated regions by methods known to those skilled in the art. For example, searches of the public databases of expressed sequences (e.g., GenBank) can identify cDNA sequences that overlap with the DST. Statistically significant sequence matches with greater than 95% nucleotide sequence matches across the overlap region can be used to generate a contiguous sequence (“contig”) and serial searches with the accumulated contig sequence can be used to assemble extended sequence associated with the DST. In cases where the assembled contig includes an open reading frame (a nucleotide sequence encoding a continuous sequence of amino acids), the polypeptide encoded by the expressed mRNA can be predicted.

In other cases, extended sequence can also be generated by making a probe containing the DST sequence. The probe would then be used to select cDNA clones by hybridization methods known in the art. These cDNA clones may be selected from libraries of cDNA clones developed from the original RNA sample, from other RNA samples, from fractionated mRNA samples, or from other widely available cDNA libraries, including those available from commercial sources. Sequences from the selected cDNA clones can be assembled into contigs in the same manner described for database sequences. The cDNA molecules can also be isolated directly from the mRNA by the rapid analysis of cDNA ends (RACE) and long range PCR. This method can be used to isolate the entire full-length cDNA or the intact 5′ and 3′ ends of the cDNA.

Methods for alignment of biological sequences for pairwise comparison are well known in the art. Local alignments between a query sequence and a subject sequence can be derived by using the algorithm of Smith (J Mol Biol, 1981), by the homology alignment algorithm of Needleman (J Mol Biol, 1970), or by the similarity search algorithm of Pearson (Proc Natl Acad Sci, 1988). A preferred method for determining the best overall match between a query sequence (a sequence of the present invention) and a subject sequence, also referred to as a sequence database, can be determined using the BLAST computer program based on the algorithm of Altschul and colleagues (Altschul, J Mol Biol; 1990; Altschul, Nucleic Acids Res, 1997). The term “sequence” includes nucleotide and amino acid sequences. In a sequence alignment, the query sequence can be either protein or nucleic acid or any combination thereof. BLAST is a statistically driven search method that finds regions of similarity between a query and database sequences. These are called segment pairs, and consist of gapless alignments of any part of two sequences. Within these aligned regions, the sum of the scoring matrix values of their constituent symbol pairs is higher than a level expected to occur by chance alone. The scores obtained in a BLAST search can be interpreted by the experienced investigator to determine real relationships versus random similarities. The BLAST program supports four different search mechanisms:
Nucleotide Query Searching a Nucleotide Database—Each database sequence is compared to the query in a separate nucleotide-nucleotide pairwise comparison.
Protein Query Searching a Protein Database—Each database sequence is compared to the query in a separate protein-protein pairwise comparison.
Nucleotide Query Searching a Protein Database—The query is translated, and each of the six products is compared to each database sequence in a separate protein-protein pairwise comparison.
Protein Query Searching a Nucleotide Database—Each nucleotide database sequence is translated, and each of the six products is compared to the query in a separate protein-protein pairwise comparison.

By using the BLAST program to search for matches between a sequence of the present invention and sequences in GenBank and EST databases, identities were assigned whenever possible. A portion of these results is listed in Table 2.

### Example 6

### DST Validation Using Real-Time Quantitative PCR

Validation of DSTs isolated by TOGA® was performed by using Real-Time Quantitative PCR using the ABI PRISM 7700 Sequence Detection System (PE Biosystems) that combines PCR, cycle-by-cycle fluorescence detection and analysis software for high-throughput quantitation of nucleic acid sequences. Reactions were characterized by the point in time when amplification of a PCR product was first detected rather than the amount of PCR product accumulated after a fixed number of cycles. The higher the copy number of the nucleic acid target, the sooner a significant increase in fluorescence is observed. Relative quantitation of the amount of target in the sample was accomplished by measuring the cycle number at which a significant amount of product was produced. The entire process was performed by the integrated software of the 7700 system. Primers for Real-Time Quantitative PCR validation were selected by the integrated software package accompanying the ABI PRISM 7700. Standards for normalizing the quantitation of gene levels were chosen from a panel of 7 mouse and 6 human “housekeeping” genes. The normalization standard chosen for the alcohol-preferring and alcohol-non-preferring rat brain samples was cyclophilin and was based on the similarity of expression across all the alcohol-preferring and alcohol-non-preferring rat brain sample templates. The results of this DST validation were compared to duplicate TOGA® runs, and the relative abundance of each DST validated in this manner is compared in Table 5. The relative abundance level of the alcohol-non-preferring brain region samples has a value of 1.00. The primers used in these studies are listed in Table 4.

FIG. 3 compares the results from Real Time PCR validation to duplicate runs of TOGA®. As the chart in FIG. 3 illustrates, the DST CAR1_{—}3 (SEQ ID NO:1) was found in a much lesser proportion in alcohol-preferring hippocampus in both TOGA® runs and Real Time PCR. The graph in FIG. 3 illustrates that, during the progressive amplification cycles of the target DST CAR1_{—}3 (SEQ ID NO:1) sequence in cortex samples, detection of the amplified sequence occurs latest in the alcohol-preferring cortex sample reflecting the fact that a lesser number of RNA molecules for that sequence were available from the start. The relative abundance is measured as compared to the alcohol-non-preferring cortex sample, which has the relative abundance set to 1.00.

### Example 7

### Extended Sequence PCR Cloning, DNA Sequence Analysis, and Gene Localization

Extended sequences for rat DSTs were generated in two ways. The most common method was to use the original DST sequence to do BLAST searches in the published database, and select those sequences with nearly 100% sequence matches. An alignment between the DST and BLAST match sequences was generated, and the 5′-most sequence was used for additional rounds of BLAST searching. Alignments between successive BLAST match sequences were used to compile a single consensus contiguous sequence (“contig”). To obtain extended rat sequences corresponding to genes encoded by the sequences of the present invention, primers corresponding to the single consensus contig for EST sequences or to known mouse sequences found within the GenBank databases were used to amplify PCR products from cDNA preparations made from adult rat hippocampus, cortex, striatum or hypothalamus from either the alcohol-preferring or alcohol-non-preferring rat samples. PCR products corresponding to extended rat sequences homologous to DSTs were gel purified, cloned into the pCR®4-TOPO plasmid vector (Invitrogen) and sequenced on both strands. Nucleotide sequences were determined by standard techniques. In order to verify that the cloned PCR product corresponds to the sequence of interest, sequences were aligned and assembled into contigs using the DNA alignment program SEQMAN (DNASTAR). The results of these analyses comprise the Extended Sequences listed in Table 6. For example, the DST CAR1_{—}3 (SEQ ID NO:1) was identified as glutathione S-transferase 8-8 based upon GenBank database BLAST results. The following results demonstrate the additional studies undertaken to establish the significance of DST CAR1_{—}3 (SEQ ID NO:1) in the pathology of alcoholism.

### Example 8

### In Situ Hybridization

Rat glutathione S-transferase 8-8 cDNA [DST CAR1_{—}3 (SEQ ID NO:1)] was cloned into the pCR®4-TOPO vector (Invitrogen, Grand Island, N.Y.). From this construct, antisense and sense cRNA probes, labeled with ³⁵S-UTP and ³⁵S-CTP, were prepared using T3 polymerase and T7 polymerase, respectively. The tissues were hybridized according to published procedures (Wirdefeldt et al, 2001). Briefly, 16-μm brain sections were dehydrated and covered with 100 μL hybridization buffer (50% formamide, 50 mM Tris, pH 8.0, 2.5 mM EDTA, pH 8.0, 50 μg/ml tRNA, 1× Denhardt's solution, 0.2 M sodium chloride, 10% Dextran sulfate) containing 1.0×10⁶ cpm labeled probe for 16 to 18 hours at 55° C. These brain sections were washed, dehydrated, and rinsed in formamide buffer (0.3 M NaCl, 50% formamide, 20 mM Tris, 1 mM EDTA pH 7.5) for 10 minutes at 60° C. After the formamide wash, the sections were treated with RNase A for 30 minutes at 37° C. The tissues were then washed in graded SSC solutions (2×SSC to 0.5 SSC) followed by dehydration in graded ethanols. The dried sections were exposed to Kodak MR film (Kodak) for 4-7 days then dipped in hypercoat LM-1 emulsion (Amersham, Piscataway, N.J.), and exposed for 2-to 3 weeks at 4° C.

Results from the in situ hybridization experiments are shown in FIG. 4. To further characterize GST 8-8 expression in P and NP rat brains, in situ hybridization was performed. Glutathione S-transferase 8-8 was widely expressed throughout the brain with expression localized to specific areas and cells. Rat GST 8-8 was highly expressed in the choroid plexus of the dorsal 3^{rd} ventricle (D3V, FIG. 4A. 1,2), the lateral ventricle (LV, FIG. 4A. 3, 4), and the CA2 and CA3 regions of the hippocampus (FIG. 4A. 3 through 6). Lower expression levels were detected in the thalamus (FIG. 4A. 4), and cortex (FIG. 4A. 6). Comparison of rGST 8-8 expression between P (FIG. 4A. 1, 3, 5) and NP (FIG. 4A. 2, 4, 6) on the autoradiographs showed lower expression in P than NP. Quantification of rGST 8-8 expression in the CA2 and CA3 regions of the P and NP hippocampus revealed a 3-fold lower expression in P (FIG. 4B. 1 and 3) than NP (FIG. 4B. 2 and 4) (p<0.0001). At the cellular level, it is clear that rGST 8-8 is expressed in the pyramidal cells of CA2 and CA3 of P and NP rat brain (FIG. 4B. 1 through 4), the endothelial cells of the choroid plexus (FIG. 4B. 5), and the ependymal cells along the dorsal third ventricle (D3V, FIG. 4B. 5). Expression of rGST 8-8 in the ependymal cells is very evident at the base of the third ventricle (3V) of NP rats (FIG. 4B. 6).

### Example 9

### Western Blot Analysis

Brain tissue from the hippocampus, striatum, amygdala, nucleus accumbens and substantia nigra was dissected from P and NP rats. Protein extracts were prepared immediately from this dissected tissue according to the Western (Immuno) Blotting protocol from Santa Cruz Biotechnology (www.scbt.com; Research Protocols). Protein concentrations were determined with the Bradford assay (Bio-Rad, Hercules, Calif.). Neuron-specific enolase (NSE; MW=45 kD) was used as the internal control to normalize the amount of neuronal protein loaded in each lane. Approximately 30 μg of the protein supernatant was fractionated on a Novex 4-20% Tris-Glycine Gel (Invitrogen, Grand Island, N.Y.), and transferred to a PVDF membrane (0.2 μm, Bio-Rad). The membrane was blocked for 1 hour in TTBS blocking buffer (5% non-fat dry milk, 1× Tris-buffered saline, 0.1% Tween-20). Different methods were used to detect rGST8-8 protein and NSE; therefore, the membrane was cut into two pieces at the 35 kD MW marker. The section of membrane containing rGST 8-8 was washed, and incubated overnight at 4° C. with the rat GST 8-8 antibody (1:1000, Cell Signaling Technology, Inc., Beverly, Mass.). After washing, the Phototope-HRP Western Blot Detection System with anti-rabbit IgG, HRP-linked antibody (1:2000, Cell Signaling Technology) was used according to the manufacturer's protocol to visualize the rGST 8-8 band. The membrane containing NSE was incubated for 1 hour with the rat NSE antisera (1:5000, Polysciences, Warrington, Pa.). After washing, the membrane was incubated for 1 hour with a HRP conjugated secondary antibody (1:15000) and the IMMUN-STAR HRP Chemiluminescent Kit (BioRad, Hercules, Calif.) was employed according to the manufacturer's protocol to detect the NSE bands. Rat GST 8-8 levels were quantified using IMAGE J software, with each lane normalized to NSE levels.

FIG. 5 illustrates rGST 8-8 protein expression analyzed using quantitative Western blot analysis of the amygdala of alcohol-preferring and alcohol-non-preferring rats. To compare rGST 8-8 protein expression in P and NP rats, five brain regions were analyzed: hippocampus, striatum, amygdala, nucleus accumbens, and substantia nigra. Similar to rGST 8-8 mRNA levels, the level of protein expression in the amygdala was lower in P rats than NP rats, with the P rats having 1.6-fold lower levels than the NP rats (FIG. 5) (p<0.020). The same trend was observed in the hippocampus, with a 1.2-fold difference between P and NP (p<0.048). No difference in expression was detected in the striatum (p<0.918), nucleus accumbens (p<0.194) or the substantia nigra (p<0.607) between the P and NP rats. It is clear from these studies that in addition to rat strain differences, there are also regional differences in the expression of rGST 8-8 in rat brain.

### Example 10

### Sequence Analysis and Identification of Single Nucleotide Polymorphisms

Reverse transcription was used to generate cDNA (RETROscript kit; Ambion, Austin, Tex.) from pooled total RNA samples that were isolated from P and NP rats. Based on the rGST 8-8 cDNA sequence (accession number: X62660), three primer pairs were designed: 2F 5′-CCAATAAGGAAACTCTGAACCAG-3′ (SEQ ID NO: 45) and 2R 5′-TTTCAAACACTGGGAAGTAACGG-3′ (SEQ ID NO: 46), 3F 5′-CTAGCTTTAGCAGTGAAGAGGG-3′ (SEQ ID NO: 47) and 3R 5′-TTTGTCATTGTTGGACAGAGTGG-3′ (SEQ ID NO: 48), 4F 5′-CCACTATGTTGACGTGGTCAG-3′ (SEQ ID NO: 49) and 4R 5′-TTAACAGTTTTTCACTCTATTTAATTGG-3′ (SEQ ID NO:50). Using P and NP brain cDNA, these primers were utilized to amplify three overlapping DNA fragments, covering the entire rGST 8-8 cDNA. Resulting PCR products were purified (GenElute PCR Cleanup Kit, Sigma, St. Louis, Mo.) and sequenced (Thermo Sequenase Cycle Sequencing Kit, USB, Cleveland, Ohio).

FIG. 6 demonstrates single nucleotide polymorphisms (SNPs) revealed by sequence analysis of the coding and 3′ untranslated region (3′ UTR) of the GST 8-8 mRNA in the two rat strains. To determine whether a sequence variation might underlie the expression difference observed between the alcohol-preferring and alcohol-non-preferring rats, the GST 8-8 cDNA was sequenced in each strain (FIG. 6). A silent single nucleotide polymorphism (SNP) was identified in the coding region at +628, relative to the translation start site (+1), where the P sequence diplayed a G to A substitution. Three SNPs were discovered in the 3′-UTR with the following substitutions at the indicated positions relative to the (+1) translation start site: a) A to G at +714, b) C to T at +727, and c) T to C at +756. Table 7 illustrates an alignment of the P and NP sequences with two published GST sequences (accession numbers X62660 and XM_{—}217195), which demonstrates that the NP GST 8-8 sequence was also more homologous to these sequences than was the P sequence in this region. Utilizing the SNP that was discovered at +727 in the iP and iNP, rGST 8-8 was mapped, using recombination-based methods, to chromosome 8, 2.9 cM distal to D8Rat21 and 3.8 cM proximal to D8Rat23, which is adjacent to the peak of the chromosome 8 QTL previously identified (Carr et al., 1998; Bice et al., 1998).

Several DSTs of the present invention are down-regulated in the alcohol-preferring rat cortex, hippocampus, striatum or hypothalamus. Furthermore these DSTs have single nucleotide polymorphisms that might be associated with the expression levels of these DSTs. Additionally, these DST gene products, such as that for DST Car 1_{—}3, may have decreased levels of protein expression that might play a role in the neurologic substrates that form the basis for alcohol seeking behavior and alcoholism. These DSTs and their derived polynucleotide and polypeptide sequences represent potential biomarkers or therapeutic targets for the diagnosis and treatment of alcoholism. For example, DST CAR1_{—}3, could be used as a diagnostic for single nucleotide polymorphisms associated with alcoholism; the DST could be used as a probe to determine differential expression of the gene as an diagnostic marker for alcohol seeking behavior, and the encoded polypeptide could be used as a therapeutic target.

The polynucleotides, polypeptides, kits and methods of the present invention may be embodied in other specific forms without department from the teachings or essential characteristics of the invention. The described embodiments are therefore to be considered in all respects as illustrative and not restrictive, the scope of the invention being indicated by the appended claims rather than the foregoing description, and all changes which come within the meaning and range of equivalency of the claims are, therefore, to be embraced within.

### LIST OF REFERENCES/PUBLICATIONS

Bice, P., Foroud, T., Bo, R., Castelluccio, P., Lumeng, L., Li, T.-K., Carr, L. G. (1998) Genomic screen for QTLs underlying alcohol consumption in the P and NP rat lines. Mamm Gen 9:949-955.
Carr, L. G., Foroud, T., Bice, P., Gobbett, T., Ivashina, J., Edenberg, H., Lumeng, L., Li, T.-K. (1998) A quantitative trait locus for alcohol consumption in selectively bred rat lines. Alcoholism: Clin. Exp. Res. 22:884-887.
Cicero T J. A critique of animal analogues of alcoholism, in Biochemistry and Pharmacology of Ethanol, Vol 2, Majchrowicz E and Noble E P, Eds., Plenum Press, New York, 533-560, 1979.
Foroud T., Li T.-K. Genetics of Alcoholism: A Review of Recent Studies in Human and Animal Models. Am. J. Addictions 8: 261-278, 1999.
Foroud, T., Bice, P., Castelluccio, P., Bo, R., Miller, L., Lumeng, L., Li, T.-K. Carr, L. G. Identification of quantitative trait loci influencing alcohol consumption in the high alcohol drinking and low alcohol drinking rat lines. Behav Genet, 30:131-140, 2000.
Froehlich J C, Harts J, Lumeng L, Li T K. Differences in response to the aversive properties of ethanol in rats selectively bred for oral ethanol preference. Pharmacol Biochem Behav 31(1):215-222, 1988.
Gatto G J, Murphy J M, Waller M B, McBride W J, Lumeng L, Li T K. Persistence of tolerance to a single dose of ethanol in the selectively-bred alcohol-preferring P rat. Pharmacol Biochem Behav 28(1):105-110, 1987.
Gatto G J, Murphy J M, Waller M B, McBride W J, Lumeng L, Li T K. Chronic ethanol tolerance through free-choice drinking in the P line of alcohol-preferring rats. Pharmacol Biochem Behav 28(1):111-115, 1987.
Koob G F. Circuits, drugs, and drug addiction. Adv Pharmacol 42:978-82, 1998.
Liang T, Spence J, Liu L, Strother W N, Chang H W, Ellison J A, Lumeng L, Li T K, Foroud T, Carr L G. alpha-Synuclein maps to a quantitative trait locus for alcohol preference and is differentially expressed in alcohol-preferring and -nonpreferring rats. Proc Natl Acad Sci USA. 2003 100, 4690-4695.
Li T-K, Lumeng L, Doolittle D P, Carr L G. Molecular associations of alcohol-seeking behavior in rat lines selectively bred for high and low voluntary ethanol drinking, Alcohol Alcohol., Suppl 1, 121-124, 1991.
McBride W J, Li T K. Animal models of Alcoholism: Neurobiology of high alcohol-drinking behavior in rodents. Crit Rev Neurobiol 12:339-369, 1998.
McBride W J. Li T K. Animal models of alcoholism: neurobiology of high alcohol-drinking behavior in rodents. Crit Rev Neurobiol. 12(4):339-69, 1998.

Murphy J M. Gatto G J, McBride W J, Lumeng L, Li T K. Operant responding for oral ethanol in the alcohol-preferring P and alcohol-non-preferring NP lines of rats. Alcohol 6(2):127-131, 1989.

TABLE 1

| | | | Relative Gene Expression Values | | | | | | | | | | | | | | | |
|---|---|---|---|---|---|---|---|---|---|---|---|---|---|---|---|---|---|---|
| Seq | | | TOGA 1 | | | | | | | | TOGA 2 | | | | | | | |
| ID | | Digital | Cortex | | Hypo | | Hippo | | Striatum | | Cortex | | Hypo | | Hippo | | Striatum | |
| No. | DST ID | Address | P | NP | P | NP | P | NP | P | NP | P | NP | P | NP | P | NP | P | NP |
| 4 | CAR1_16 | ACAT277 | 1531 | 598 | 2106 | 461 | 1312 | 436 | 3651 | 464 | 1982 | 543 | 2946 | ND | 1193 | 574 | 4760 | 939 |
| 5 | CAR1_17 | AGAC141 | 112 | 1584 | 372 | 924 | 219 | 1139 | 96 | 162 | 119 | 1275 | 418 | ND | 167 | 923 | 65 | 130 |
| 7 | CAR1_30 | ATCG411 | 21 | 132 | 35 | 810 | 25 | 303 | 38 | 491 | 41 | 432 | 50 | ND | 22 | 343 | 32 | 904 |
| 1 | CAR1_3 | ATGG266 | 335 | 980 | 414 | 1366 | 302 | 932 | 115 | 547 | 108 | 888 | 81 | ND | 129 | 820 | 153 | 1329 |
| 2 | CAR1_5 | GATT405 | 302 | 97 | 208 | 62 | 295 | 79 | 88 | 33 | 301 | 66 | 382 | ND | 401 | 82 | 110 | 44 |
| 3 | CAR1_7 | GTAG286 | 2367 | 2090 | 2844 | 1878 | 3196 | 1368 | 1320 | 2022 | 2806 | 2180 | 1734 | ND | 3287 | 1314 | 2120 | 1375 |
| 6 | CAR1_28 | TGAT123 | 27 | 2607 | 286 | 1284 | 53 | 1293 | 281 | 1398 | 124 | 2415 | 405 | ND | 204 | 1406 | 199 | 1289 |

TABLE 2

| | | Digital | | | Nucleotide identity | |
|---|---|---|---|---|---|---|
| Seq | | Address | | | DST nucleotide | Database nucleotide |
| ID | DST ID | (Msp1) | Database Match (Accession #) | % Identity | range (bp#) | range (bp#) |
| 3 | CAR1_7 | GTAG 286 | EST Normalized rat brain, Bento Soares | 95% | 1-233 | 5-237 |
| | | | Rattus sp. cDNA clone RBRCT09 3 end | | | |
| | | | (A1228307.1) | | | |
| 4 | CAR1_16 | ACAT 277 | Rattus norvegicus rS-Rex-b mRNA, | 96% | 1-225 | 2763-2986 |
| | | | complete cds (U176041.1) or | | | |
| | | | Rat mRNA for C1-13 gene product | 96% | 1-225 | 729-953 |
| | | | (X52817.1) | | | |
| 5 | CAR1_17 | AGAC 141 | EST UI-R-BO0-ahl-g-10-0-UI.s1 UI-R- | 98% | 1-88 | 13-100 |
| | | | BO0 Rattus norvegicus cDNA clone UI-R- | | | |
| | | | BO0-ahl-g-10-0-UI 3 (AW522737.1) | | | |
| 6 | CAR1_28 | TGAT 123 | EST UI-R-BO0-aha-a-02-0-UI.s1 UI-R- | 97% | 1-72 | 11-82 |
| | | | BO0 Rattus norvegicus cDNA clone UI-R- | | | |
| | | | BO0-aha-a-02-0-UI 3′, mRNA sequence | | | |
| 7 | CAR1_30 | ATCG 411 | EST UI-R-AB0-vq-b-11-0-UI.s1 UI-R- | 98% | 2-349 | 157-504 |
| | | | AB0 Rattus norvegicus cDNA clone UI-R- | | | |
| | | | AB0-vq-b-11-0-UI 3 (AI575571.1) | | | |

EST = Expressed Sequence Tag,
N/A = Not Applicable

TABLE 3

| | | Digital | |
|---|---|---|---|
| SEQ | | Address | |
| ID NO | DST ID | (Msp1) | Gene Identity (Accession #) |
| 1 | CAR1_3 | ATGG 266 | R. rattus mRNA for glutathione |
| | | | transferase subunit 8 (X62660) |
| 2 | CAR1_5 | GATT 405 | EST to UI-R-BJ0-aeb-c-12-0-UI.s1 |
| | | | UI-R-BJ0 Rattus norvegicus cDNA |
| | | | clone (AW252680.1) |

TABLE 4

| SEQ | | Digital | | SEQ | | SEQ |
|---|---|---|---|---|---|---|
| ID | | Address | | ID | | ID |
| NO | DST ID | (Msp1) | Forward Primer | NO | Reverse Primer | NO |
| 1 | CAR1_3 | ATGG 266 | ACTGCAAGGGTCCAATCACAG | 31 | TCTTGCCTCTGGAATGCTCTG | 32 |
| 2 | CAR1_5 | GATT 405 | GTGTGGTGGCCTTGTCTGG | 33 | TGGCTGTCACCAAAGAGCAG | 34 |
| 3 | CAR1_7 | GTAG 286 | TTCAGGACCAGAATGAGGCG | 35 | AAGTGCAAGCTGGCAGCAA | 36 |
| 4 | CAR1_16 | ACAT 277 | TGATTGTTTCCCTTCCCCAG | 37 | TGATTGTTTCCCTTCCCCAG | 38 |
| 5 | CAR1_17 | AGAC 141 | GGAGACCACGGGAAATTGCT | 39 | CCTAGGGCTCTCAGCTCGCT | 40 |
| 6 | CAR1_28 | TGAT 123 | GATGTACCACTAACAGGGATTTAGGG | 41 | GTGGATCCTTAGGTAAATAGGCCA | 42 |
| 7 | CAR1_30 | ATCG 411 | GAACGTGCCCCTTCCTCATA | 43 | TCAACTCTGCGTCTCTTGCTGT | 44 |

TABLE 5
Real-Time PCR Validation

| Seq | | Digital | | | | | | | |
|---|---|---|---|---|---|---|---|---|---|
| ID | | Address | | TOGA-1 | | TOGA-2 | | Real-Time PCR | |
| No | DST ID | (MspI) | Tissue | NP | P | NP | P | NP | P |
| 1 | CAR1_3 | ATGG 266 | Cortex | 1.00 | 0.34 | 1.00 | 0.12 | 1.00 | 0.30 |
| | | | Hypothalamus | 1.00 | 0.30 | ND | ND | 1.00 | 0.33 |
| | | | Striatum | 1.00 | 0.21 | 1.00 | 0.12 | 1.00 | 0.06 |
| | | | Hippocampus | 1.00 | 0.32 | 1.00 | 0.16 | 1.00 | 0.12 |
| 2 | CAR1_5 | GATT 405 | Cortex | 1.00 | 3.11 | 1.00 | 4.56 | 1.00 | 1.13 |
| | | | Hypothalamus | 1.00 | 3.35 | ND | ND | 1.00 | 1.07 |
| | | | Striatum | 1.00 | 2.67 | 1.00 | 2.50 | 1.00 | 0.94 |
| | | | Hippocampus | 1.00 | 3.73 | 1.00 | 4.89 | 1.00 | 3.07 |
| 3 | CAR1_7 | GTAG 286 | Cortex | 1.00 | 1.13 | 1.00 | 1.29 | 1.00 | 18.9 |
| | | | Hypothalamus | 1.00 | 1.51 | ND | ND | 1.00 | 11.37 |
| | | | Striatum | 1.00 | 0.65 | 1.00 | 1.54 | 1.00 | 24.1 |
| | | | Hippocampus | 1.00 | 2.34 | 1.00 | 2.50 | 1.00 | 9.27 |
| 4 | CAR1_16 | ACAT 277 | Cortex | 1.00 | 2.56 | 1.00 | 3.65 | 1.00 | 1.42 |
| | | | Hypothalamus | 1.00 | 4.57 | ND | ND | 1.00 | 1.52 |
| | | | Striatum | 1.00 | 7.87 | 1.00 | 5.07 | 1.00 | 1.58 |
| | | | Hippocampus | 1.00 | 3.01 | 1.00 | 2.08 | 1.00 | 3.85 |
| 5 | CAR1_17 | AGAC 141 | Cortex | 1.00 | 0.07 | 1.00 | 0.09 | 1.00 | 0.29 |
| | | | Hypothalamus | 1.00 | 0.40 | ND | ND | 1.00 | 0.38 |
| | | | Striatum | 1.00 | 0.59 | 1.00 | 0.50 | 1.00 | 0.35 |
| | | | Hippocampus | 1.00 | 0.19 | 1.00 | 0.18 | 1.00 | 0.3 |
| 6 | CAR1_28 | TGAT 123 | Cortex | 1.00 | 0.01 | 1.00 | 0.05 | 1.00 | 0.13 |
| | | | Hypothalamus | 1.00 | 0.22 | ND | ND | 1.00 | 0.33 |
| | | | Striatum | 1.00 | 0.20 | 1.00 | 0.15 | 1.00 | 0.16 |
| | | | Hippocampus | 1.00 | 0.04 | 1.00 | 0.15 | 1.00 | 0.14 |
| 7 | CAR1_30 | ATCG 411 | Cortex | 1.00 | 0.16 | 1.00 | 0.09 | 1.00 | 0.55 |
| | | | Hypothalamus | 1.00 | 0.04 | ND | ND | 1.00 | 0.58 |
| | | | Striatum | 1.00 | 0.08 | 1.00 | 0.04 | 1.00 | 0.70 |
| | | | Hippocampus | 1.00 | 0.08 | 1.00 | 0.06 | 1.00 | 0.80 |

TABLE 6

| DST ID | DST Seq ID NO | EXT Seq ID NO |
|---|---|---|
| CAR1_3 | 1 | 8 |
| CAR1_7 | 3 | 9 |
| CAR1_16 | 4 | 10, 11 |
| CAR1_17 | 5 | 12 |
| CAR1_28 | 6 | 13 |

TABLE 7
Nucleotide position of polymorphisms in GST 8—8 sequences

| Name | 628 | 714 | 727 | 756 | 781 | 799 |
|---|---|---|---|---|---|---|
| rGST 8—8 P | A | G | T | C | CTGA | A |
| rGST 8—8 NP | G | A | C | T | CTGA | A |
| X62660 | G | A | C | T | CTGA | A |
| XM_217195 | G | A | C | T | G | Deletion |

We claim:

1. A method for preventing or treating alcoholism or ameliorating a symptom of alcoholism, comprising administering to a mammalian subject a therapeutically effective amount of at least one of:
(a) a first polynucleotide chosen from the group consisting of SEQ ID NOs: 1-14, or a second polynucleotide at least 95% identical to said first polynucleotide; or
(b) a third polynucleotide at least ten bases in length that is hybridizable to said first polynucleotide under stringent conditions; or
(c) a gene corresponding to any of the foregoing polynucleotides, another gene at least 95% identical to said gene, or a region of any of the foregoing genes,
wherein the symptom of alcoholism is selected from the group consisting of fatty liver, alcoholic hepatitis, fibrosis, cirrhosis, hypertension, weakened heart muscle, and arrhythmia.

2. A method for preventing or treating alcoholism or ameliorating a symptom of alcoholism, comprising administering to a mammalian subject a therapeutically effective amount of:
(a) a first polypeptide encoded by a polynucleotide chosen from the group consisting of SEQ ID NOs: 1-14 or another polynucleotide at least 95% identical to said polynucleotide; or
(b) a second polypeptide encoded by a gene corresponding to any of the foregoing polynucleotides or another gene at least 95% identical to said gene; or
(c) a third polypeptide at least 90% identical to one of the foregoing polypeptides; or
(d) a fragment of one of the foregoing polypeptides,
wherein the symptom of alcoholism is selected from the group consisting of fatty liver, alcoholic hepatitis, fibrosis, cirrhosis, hypertension, weakened heart muscle, and arrhythmia.

3. A method for preventing or treating alcoholism or ameliorating a symptom of alcoholism, comprising administering to a mammalian subject a therapeutically effective amount of an antibody that binds specifically to at least one of:
(a) a first polypeptide encoded by a polynucleotide chosen from the group consisting of SEQ ID NOs: 1-14, or another polynucleotide at least 95% identical to said polynucleotide; or
(b) a second polypeptide encoded by a gene corresponding to any of the foregoing polynucleotides or another gene at least 95% identical to said gene; or
(c) a third polypeptide at least 90% identical to any one of the foregoing polypeptides; or
(d) a fragment of any one of the foregoing polypeptides,
wherein the symptom of alcoholism is selected from the group consisting of fatty liver, alcoholic hepatitis, fibrosis, cirrhosis, hypertension, weakened heart muscle, and arrhythmia.

4. A method for assessing the efficacy of a test compound for treating alcoholism in a mammalian subject, the method comprising the step of comparing:
(a) a level of expression of a marker in a first sample obtained from the subject, wherein the first sample is exposed to the test compound and wherein the marker is selected from the group consisting of polynucleotides listed in SEQ ID NO: 1-14; polypeptides encoded by the polynucleotides listed in SEQ ID NO: 1-14; and fragments thereof; and
(b) a level of expression of the same marker in a second sample obtained from the subject, wherein the second sample is not exposed to the test compound, and wherein a substantially increased or decreased level of expression of the marker in the first sample, relative to the second sample, is an indication that the test compound is efficacious in treating alcoholism.

5. A method for diagnosing alcoholism or a susceptibility to alcoholism in a subject comprising:
(a) determining the presence of a mutation in:
(i) a first polynucleotide chosen from the group consisting of SEQ ID NOs: 1-14, or a second polynucleotide at least 95% identical to said first polynucleotide; or
(ii) a third polynucleotide at least ten bases in length that is hybridizable to said first polynucleotide under stringent conditions; or
(iii) a gene corresponding to any of the foregoing polynucleotides, another gene at least 95% identical to said gene, or a region of any of the foregoing genes; and
(b) diagnosing alcoholism or a susceptibility to alcoholism based on the presence of said mutation.

6. A method for diagnosing alcoholism or a susceptibility to alcoholism in a subject, the method comprising:
(a) obtaining a first biological sample from a patient suspected of having alcoholism;
(b) obtaining a second sample from a suitable comparable control source;
(c) determining in the first and second samples a level of expression of at least one polynucleotide selected from the group consisting of:
(i) a first polynucleotide chosen from the group consisting of SEQ ID NOs: 1-14;
(ii) a second polynucleotide at least 95% identical to said first polynucleotide;
(iii) a third polynucleotide at least ten bases in length that is hybridizable to said first polynucleotide under stringent conditions; and
(iv) a gene corresponding to any of the foregoing polynucleotides, another gene at least 95% identical to said gene, or a region of any of the foregoing genes;
(d) comparing in the first and second samples the level of expression of the at least one polynucleotide, wherein a patient is diagnosed as having or susceptible to alcoholism if the amount of the at least one polynucleotide molecule in the first sample is greater than or less than the amount of the at least one polynucleotide molecule in the second sample.

7. A method for diagnosing alcoholism or a susceptibility to alcoholism in a subject, the method comprising:
(a) obtaining a first biological sample from a patient suspected of having alcoholism;
(b) obtaining a second sample from a suitable comparable control source;
(c) determining in the first and second samples a level of expression of at least one polypeptide selected from the group consisting of:
(i) a first polypeptide encoded by a polynucleotide chosen from the group consisting of SEQ ID NOs: 1-14 or another polynucleotide at least 95% identical to said polynucleotide;
(ii) a second polypeptide encoded by a gene corresponding to any of the foregoing polynucleotides or another gene at least 95% identical to said gene;
(iii) a third polypeptide at least 90% identical to one of the foregoing polypeptides; and
(iv) a fragment of one of the foregoing polypeptides;
(d) comparing the level of expression of the at least one polypeptide in the first and second samples, wherein a patient is diagnosed as having or susceptible to alcoholism if the amount of the at least one polypeptide in the first sample is greater than or less than the amount of the at least one polypeptide in the second sample.

8. A method for assessing a stage of alcoholism by testing for regulation of at least one of:
(a) a first polynucleotide chosen from the group consisting of SEQ ID NOs: I-14, or a second polynucleotide at least 95% identical to said first polynucleotide; or
(b) a third polynucleotide at least ten bases in length that is hybridizable to said first polynucleotide under stringent conditions; or
(c) a gene corresponding to any of the foregoing polynucleotides, another gene at least 95% identical to said gene, or a region of any of the foregoing genes.

9. A method for assessing the efficacy or toxicity of a therapeutic treatment for alcoholism by testing for regulation of at least one of:
(a) a first polynucleotide chosen from the group consisting of SEQ ID NOs: 1-14, or a second polynucleotide at least 95% identical to said first polynucleotide; or
(b) a third polynucleotide at least ten bases in length that is hybridizable to said first polynucleotide under stringent conditions; or
(c) a gene corresponding to any of the foregoing polynucleotides, another gene at least 95% identical to said gene, or a region of any of the foregoing genes.

10. A method for assessing a stage of alcoholism by testing for regulation of at least one of:
(a) a first polypeptide encoded by a polynucleotide chosen from the group consisting of SEQ ID NOs: 1-14 or another polynucleotide at least 95% identical to said polynucleotide; or
(b) a second polypeptide encoded by a gene corresponding to any of the foregoing polynucleotides or another gene at least 95% identical to said gene; or
(c) a third polypeptide at least 90% identical to one of the foregoing polypeptides; or
(d) a fragment of one of the foregoing polypeptides.

11. A method for identifying a binding partner comprising:
(a) contacting at least one polypeptide with a binding partner, wherein the at least one polypeptide is selected from the group consisting of:
(i) a first polypeptide encoded by a polynucleotide chosen from the group consisting of SEQ ID NOs: 1-14 or another polynucleotide at least 95% identical to said polynucleotide;
(ii) a second polypeptide encoded by a gene corresponding to any of the foregoing polynucleotides or another gene at least 95% identical to said gene;
(iii) a third polypeptide at least 90% identical to one of the foregoing polypeptides; and
(iv) a fragment of one of the foregoing polypeptides; and
(b) determining whether the binding partner affects an activity of said polypeptide.

12. A method for identifying a binding partner, the method comprising:
(a) contacting at least one polypeptide with a binding partner, wherein the at least one polypeptide is selected from the group consisting of:
(i) a first polypeptide encoded by a polynucleotide chosen from the group consisting of SEQ ID NOs: 1-14, or another polynucleotide at least 95% identical to said polynucleotide;
(ii) a second polypeptide encoded by a gene corresponding to any of the foregoing polynucleotides or another gene at least 95% identical to said gene;
(iii) a third polypeptide at least 90% identical to any one of the foregoing polypeptides; and
(iv) a fragment of any one of the foregoing polypeptides;
(b) contacting the at least one polypeptide bound to the binding partner with an antibody capable of immunospecifically binding to the at least one polypeptide bound to the binding partner.

13. A first substantially pure isolated DNA molecule suitable for use as a probe for genes regulated in alcoholism, wherein said first substantially pure isolated DNA molecule is a polynucleotide chosen from the group consisting of SEQ ID NO:1-14, a gene corresponding to said polynucleotide, or regions of said gene; or a second substantially pure isolated DNA molecule at least 95% similar to said first substantially pure isolated DNA molecule.

14. A kit for detecting in a sample from a mammalian subject the presence of at least one polypeptide encoded by:
(a) a first polynucleotide chosen from the group consisting of SEQ ID NOs: 1-14, or a second polynucleotide at least 95% identical to said first polynucleotide; or
(b) a third polynucleotide at least ten bases in length that is hybridizable to said first polynucleotide under stringent conditions; or
(c) a gene corresponding to any of the foregoing polynucleotides, another gene at least 95% identical to said gene, or a region of any of the foregoing genes,
wherein said kit comprises a biomolecule which specifically binds with said at least one polypeptide in an amount sufficient for at least one assay and suitable packaging material.

15. The kit of claim 14 wherein the biomolecule is a first antibody.

16. The kit of claim 15 further comprising a second antibody that binds to the first antibody, wherein the second antibody is labeled.

17. A kit for detecting the presence of a gene encoding a protein comprising a first polynucleotide chosen from the group consisting of SEQ ID NO:1-14, or a second polynucleotide at least 95% identical to said first polynucleotide, or a third polynucleotide at least ten bases in length that is hybridizable to said first polynucleotide under stringent conditions, or a fragment of any of the foregoing polynucleotides having at least 10 contiguous bases, in an amount sufficient for at least one assay, and suitable packaging material.

18. A method for detecting the presence of a nucleic acid encoding a protein in a mammalian subject, comprising the steps of:
(a) obtaining a biological sample from the subject;
(b) hybridizing with a first polynucleotide from the sample or a first gene corresponding to said first polynucleotide:
(i) a second polynucleotide chosen from the group consisting of SEQ ID NO:1-14 or a second gene corresponding to said second polynucleotide; or
(ii) a third polynucleotide at least 95% identical to said second polynucleotide or a third gene corresponding to said third polynucleotide; or
(iii) a fourth polynucleotide at least ten bases in length that is hybridizable to said second polynucleotide under stringent conditions or a fourth gene corresponding to said fourth polynucleotide; or
(iv) a fragment of any of the foregoing polynucleotides or any of the foregoing genes having at least 10 contiguous bases; and
(c) detecting the presence of the hybridization product.

19. A method for providing a therapeutic molecule to a mammalian subject afflicted with alcoholism and in need of a therapeutic molecule, the method comprising:
(a) linking the therapeutic molecule to a polynucleotide selected from the group consisting of:
(i) a first polynucleotide identified in SEQ ID NOs: 1-14;
(ii) a second polynucleotide at least 95% identical to said first polynucleotide;
(iii) a third polynucleotide at least ten bases in length that is hybridizable to said first polynucleotide under stringent conditions; and
(iv) a gene corresponding to any of the foregoing polynucleotides, another gene at least 95% identical to said gene, or a region of any of the foregoing genes; and
(b) administering the therapeutic molecule linked to the polynucleotide to the mammalian subject, wherein the therapeutic molecule is selected from the group consisting of genes, vaccines, diagnostic reagents, peptides, proteins and macromolecules.

20. A method for providing a therapeutic molecule to a mammalian subject afflicted with alcoholism and in need of the therapeutic molecule, the method comprising:
(a) linking the therapeutic molecule to a polypeptide selected from the group consisting of:
(i) a first polypeptide encoded by a polynucleotide identified in SEQ ID NOs: 1-14, or another polynucleotide at least 95% identical to said polynucleotide;
(ii) a second polypeptide encoded by a gene corresponding to any of the foregoing polynucleotides or another gene at least 95% identical to said gene;
(iii) a third polypeptide at least 90% identical to any one of the foregoing polypeptides; and
(iv) a fragment of any one of the foregoing polypeptides; and
(b) administering the therapeutic molecule linked to the polypeptide to the mammalian subject, wherein the therapeutic molecule is selected from the group consisting of genes, vaccines, diagnostic reagents, peptides, proteins and macromolecules.

21. A method for providing a therapeutic molecule to a mammalian subject afflicted with alcoholism and in need of the therapeutic molecule, the method comprising:
(a) linking the therapeutic molecule to an antibody capable of immunospecific binding to a polypeptide selected from the group consisting of:
(i) a first polypeptide encoded by a polynucleotide identified in SEQ ID NOs: 1-14, or another polynucleotide at least 95% identical to said polynucleotide;
(ii) a second polypeptide encoded by a gene corresponding to any of the foregoing polynucleotides or another gene at least 95% identical to said gene;
(iii) a third polypeptide at least 90% identical to any one of the foregoing polypeptides; and
(iv) a fragment of any one of the foregoing polypeptides; and
(b) administering the therapeutic molecule linked to the antibody to the mammalian subject, wherein the therapeutic molecule is selected from the group consisting of genes, vaccines, diagnostic reagents, peptides, proteins and macromolecules.

22. A method for predicting whether a subject afflicted with alcoholism is likely to respond favorably to a treatment prior to administration of the treatment to the subject, the method comprising the steps of:
(a) obtaining a sample from the subject;
(b) determining a level of expression of at least one of:
(i) a first polynucleotide selected from the group consisting of SEQ ID NOs: 1-14;
(ii) a second polynucleotide at least 95% identical to the first polynucleotide;
(iii) a gene corresponding to any of the foregoing polynucleotides, another gene at least 95% identical to the gene, or a region of any of the foregoing genes;
(iv) a first polypeptide encoded by a polynucleotide selected from the group consisting of SEQ ID NOs: 1-14;
(v) a second polypeptide encoded by a gene corresponding to any of the foregoing polynucleotides or another gene at least 95% identical to said gene;
(vi) a third polypeptide at least 90% identical to one of the foregoing polypeptides; or
(vii) a fragment of one of the foregoing polypeptides; and
(c) comparing the level of expression to a database comprising expression patterns from patients previously given the treatment,
wherein a similar level of expression from the subject as compared to the level of expression from the database of patients that responded favorably to the treatment predicts that the subject will respond favorably to the treatment.


SEQUENCE LISTING (50 sequences)

SEQ ID NO: 1 - DNA, 217 bp, Rattus norvegicus
cggatggccactatgttgacgtggtcaggaccgtcctgaagttctagtgacagcgtgctttaaagtggctactgcaagggtccaatcacagcagcagctacagagcattccagaggcaagatagagctctcaggagtaaaggtcttcaaagaacctgaaaaccactctgtccaacaatgacaaatgccaattaaatagagtgaaaaactgttaaaaa

SEQ ID NO: 2 - DNA, 356 bp, Rattus norvegicus
cgggattcagtcatgcctgtctcagttaacttgtaaaactattaaagttccagaaatcgggccattcacttaatgtccaatgtggacttcttattaatatgacagtcagttaccaagacgtcagttaggagtgtggtggccttgtctgggcttttgtcactctgctctttggtgacagccactgtagtccaggatcatatccctcatggcctagaactgtgtagcccaggctgacttcaaatttatggtcttcctgcttcaaactcctatatcctgggatttagcatttgtcatggtctaggtcactttgtatatagaactttgttgtggtcaataaacctattctgagggaaaaa

SEQ ID NO: 3 - DNA, 235 bp, Rattus norvegicus
cgggtaggtagggasccgtggtcacactgggctggcaagccatcttctgggagacttgaactttcaggaccagaatgaggcggcggcaggaggattgctgccagcttgcactttctaattttagagttttttttttaaaaagatcatataaacggggtctaaaaaatggtctcttgtatccttgtgtttgataatagtttaatatgcttgagcaataaatgctaacacgcaaaaa

SEQ ID NO: 4 - DNA, 225 bp, Rattus norvegicus
cggacatgaatgggggtgtctggagtggtaacagctctctccttcctcgttactgcgaattgattgtttcccttccccagtaccataatcttagaggcaaacttcgagacaggtgttttcagtcttttcctgtactcttaggatatttgagtcccccccgccccttgtcaagcactaaagtatagagtgtgttagatgtggtttttaattttgtgttgctaaaaa

SEQ ID NO: 5 - DNA, 88 bp, Rattus norvegicus
cggagaccacgggaaattgctcaaggcaccagagcgagctgagagccctagggccaacagtcaataaaagtccattgccaccgaaaaa

SEQ ID NO: 6 - DNA, 72 bp, Rattus norvegicus
cggtgatgtaccactaacagggatttagggttggcctatttacctaaggatccacaggttgaacgccaaaaa

SEQ ID NO: 7 - DNA, 360 bp, Rattus norvegicus
  misc_feature (350)..(350): n is a, c, g, or t
cggatcgagagcggaacgtgccccttcctcatagaacaacaaacagcaagagacgcagagttgagagaaggagtccagtgagctcccctcgggccttaaaagcacagactcgtattatttacaaaagtaggcctgggacaagttcatgctccgagaggacatcccttcaaggttgggctccgattcctgcatcttgggatctaggcccgtggccacaggtgcctgtcggtgtgcactgccaagagacaccacaccgctaggaaagaagagttcatcaatcactctcttgggagtgtttgggacagaggagaggtgattatccagattacctgaacgacaagactttgaancanagaaaaa

SEQ ID NO: 8 - DNA, 893 bp, Rattus norvegicus
ccaataaggaaactctgaaccaggagtcatggaagtcaaacccaagctctactactttcaaggcaggggaaggatggagtcgatccgctggctgctggctacagctggagtggagtttgaagaagaatttcttgagacgagagaacaatatgagaagttgcaaaaggatggatgcctgctttttggccaagtcccattggtggaaatagacgggatgctactgacacagaccagagccatcctcagctacctggccgccaagtacaacttgtatgggaaggacctgaaggagagagtcaggattgacatgtatgccgatggcacccaggacctgatgatgatgattatcggggctccatttaaagcccctcaggaaaaagaagagagcctagctttagcagtgaagagggctaaaaaccgttacttcccagtgtttgaaaagattttaaaagaccatggagaggcatttcttgttggcaaccaactcagttgggcagacatacagctactagaagccattttgatggtggaagaagtcagtgctcctgtgttgtctgacttccctctgctgcaggcatttaagacaagaatcagcaacattcctacaattaagaagttcctgcaacctggaagtcagaggaagccacctccggatggccactatgttgacgtggtcaggaccgtcctgaagttctagtgacagcgtgctttaaagtggctactgcaagggtccaatcacagcagcagctacagagcattccagaggcaagatagagctctcaggagtaaaggtcttcaaagaacctgaaaaccactctgtccaacaatgacaaatgccaattaaatagaggaaaaactgttaaaaaaaaaaaaaaaaaaaaaaaaaaaaaaaa

SEQ ID NO: 9 - DNA, 704 bp, Rattus norvegicus
tcttgggctggggcggacagtgtacgggatggaggcgactttggagcagcatttggaggacacaatgaagaatccatccattgttggagtcctatgcacagattcacaaggacttaatctgggctgccgtggtaccctgtcggatgagcatgctggagtgatatctgttctcgcccagcaggcagctaagctgacctctgaccccaccgacatccctgtagtgtgtttagagtcagataacgggaatgttatgatccagaaacacgatggcatcacagtggctgtgcacaaaatggcctcttgacatctgatgccagctctccagtggtctcccaccgggattcagtcatgcctgtctcagttaacttgtaaaactattaaagttccagaaatcgggccattcacttaatgtccaatgtggacttcttattaatatgacagtcagttaccaagacgtcagttaggagtgtggtggccttgtctgggcttttgtcactctgctctttggtgacagccactgtagtccaggatcatatccctcatggcctagaactgtgtagcccaggctgacttcaaatttatggtcttcctgcttcaaactcctatatcctgggatttagcatttgtcatggtctaggtcactttgtatatagaactttgttgtggtcaataaacctattctgagggaaaaaaaaaaaaaaaaa

SEQ ID NO: 10 - DNA, 807 bp, Rattus norvegicus
tgcaagctcagcgactggaggagagccacgagcccgccaagcccgcaccatgcagccactgccgattccaccaagatggactgtgtgtggagcaactggaaaagtcaggctattgaccttctgtactggcgggacatcaagcagacggggattgtgttcgggagcttcctgctgctgctcttctccctgacccagttcagcgttgtgagcgtcgtcgcctacctggccctggctgccctctctgccaccatcagcttccgcatctacaagtccgttctacaagctgtgcagaaaacagatgagggtcaccctttcaaggcctacctggagctggagatcaccctgtcccaggagcagatccagaagtacacagactgcctgcagctatacgtgaacagcactctgaaggagctacggaggctcttccttgtccaagacctagtggattccttaaaatttgcagtcctcatgtggctcctgacctacgttggcgcactcttcaatggcctgaccctgctgcttatggctgtggtttcgatgtttactctacctgtggtatatgttaagcaccaggcacaggttgaccaatatctgggacttgtgaggactcacataaacaccgttgtggcaaagatccaggctaaaatccccggcgcaagaggcatgctgagtagatggttgccccaggagaagccggacatgaatgggggtgtctggagtggtaacagctctctccttcctcgttactgcgaattgattgtttcccttccccagtaccataatcttagaggcaaacttcgagacagg

SEQ ID NO: 11 - DNA, 1198 bp, Rattus norvegicus
cgctaccctctggctatgtgagcttcggccacgtgtcgggcccgcctccctctcctgcctctccatctatccagtacagcatcctgcgggaggagcgggaggccgagctggacagcgagctcatcatcgagtcctgcratgcctcctcggcctcagaggagagccccaagcgcgagcaggactcgcccccgatgaaaccgggcgtcctggatgccatccgcgaagagacaagctctcgggccaccgaagagcgagcgcccagccaccaaggccctgtggaacctgacccgatactcagcttcacccctgtcaccctccagtcccgcccagagccatcatcaggtgatggagccccggtcccagagccgcccaagtcacagcagcagaagcctgaagaggaagctgtgagttccagccaaagtcccgcggccaccgaaattcctggaccacttggttctgacctcgtgccccctctgcctttcttcaacaagcaaaaagctattgaccttctgtactggcgggacatcaagcagacggggattgtgttcgggagcttcctgctgctgctcttctccctgacccagttcagcgttgtgagcgtcgtcgcctacctggccctggctgccctctctgccaccatcagcttccgcatctacaagtccgttctacaagctgtgcagaaaacagatgagggtcaccctttcaaggcctacctggagctggagatcaccctgtcccaggagcagatccagaagtacacagactgcctgcagctatacgtgaacagcactctgaaggagctacggaggctcttcctggtccaggacctggtggattccttaaaatttgcagtcctcatgtggctcctgacctacgttggcgcactcttcaatggcctgaccctgctgcttatggctgtggtttcgatgtttactctacctgtggtatatgttaagcaccaggcacaggttgaccaatatctgggacttgtgaggactcacataaacaccgttgtggcaaagatccaggctaaaatccccggcgccaagaggcaygctgagtagatggttgccccaggagaagccggacatgaatgggggtgtctggagtggtaacagctctctccttcctcgttactgcgaattgattgtttcccttccccagtaccataatacttagaggcaaacttcgagacagg

SEQ ID NO: 12 - DNA, 398 bp, Rattus norvegicus
gagcaatacgcccacgctgtgcgcggccacttgcacaacgttgaccagctcttgaccctgcaacgacgcaggctcagcctcctggaggaaaattacaacatggagctggaggtcctaaccaaagagtttgagaccgaaaggaaagcaatccttgaccatcacgataaggaaatccactacctgcacgacgtcttcatggccatggagcagaactatgtggactcggagtatgaaagcaagctagaattccagagcatgtgggatgacctgaaaaacaaggtatagaaggggacgggtccggagaccacgggaaattgctcaaggcaccagagcgagctgagagccctagggccaacagtcaataaaagtccattgccaccgaaaaaaaaaaaaaaaaa

SEQ ID NO: 13 - DNA, 545 bp, Rattus norvegicus
cacgaggggaagaagcccatgccagccctgccaggggaccttgaggcatggagtctcgcggggacccctagcccgacctccggtctggtccccaggaacagcaagcagcaagcagcaagcagggcgggctgcagccccagagcagcagaatcaagtargatcctgtgtgtgccgcgtcgttcctgctggcgagacgtagcgcgggacatctggtgccaaaaacccgggaaagaccttgccatcgtcagcaccttcggagatcccttggcgacaaggaggattcagaactgcaggacaatgaaaatcccatatggatcccaggacgacttactcggagcatcacagcaagtgatcaagaagggactgggactcctgcccctcctcactcttcttccatggatgccagcaacggtgcagggcgagctacgatggggaataatgtcaacctttccactcccgatgccggtgatgtaccactaacagggatttagggttggcctatttacctaaggatccacaggttgaacgmcaaaaaaaaaaaaaaa

SEQ ID NO: 14 - DNA, 48 bp, Artificial Sequence
  Synthetic Construct
gaattcaactggaagcggccgcaggaattttttttttttttttttvnn

SEQ ID NO: 15 - DNA, 16 bp, Artificial Sequence
  Synthetic Construct
aggtcgacggtatcgg

SEQ ID NO: 16 - DNA, 16 bp, Artificial Sequence
  Synthetic Construct
ggtcgacggtatcggn

SEQ ID NO: 17 - DNA, 15 bp, Artificial Sequence
  Synthetic Construct
gagctccaccgcggt

SEQ ID NO: 18 - DNA, 16 bp, Artificial Sequence
  Synthetic Construct
cgacggtatcggnnnn

SEQ ID NO: 19 - DNA, 77 bp, Artificial Sequence
  Synthetic Construct
gaattcaactggaagcggccgcaggaagagctccaccgcggtagtactcactgcagttttttttttttttttttvnn

SEQ ID NO: 20 - DNA, 50 bp, Artificial Sequence
  Synthetic Construct
aattcggtaccaattaaccctcactaaagggacctcgaggtcgacggtat

SEQ ID NO: 21 - DNA, 52 bp, Artificial Sequence
  Synthetic Construct
cgataccgtcgacctcgaggtccctttagtgagggttaattggtaccgaatt

SEQ ID NO: 22 - DNA, 15 bp, Artificial Sequence
  Synthetic Construct
gagctccaccgcggt

SEQ ID NO: 23 - DNA, 22 bp, Artificial Sequence
  Synthetic Construct
cctcgaggtcgacggtatcggn

SEQ ID NO: 24 - DNA, 16 bp, Artificial Sequence
  Synthetic Construct
cgacggtatcggatgg

SEQ ID NO: 25 - DNA, 30 bp, Artificial Sequence
  Synthetic Construct
gatcgaatccggatggccactatgttgacg

SEQ ID NO: 26 - DNA, 59 bp, Artificial Sequence
  Synthetic Construct
tcccagtcacgacgttgtaaaacgacggctcatatgaattaggtgaccgacggtatcgg

SEQ ID NO: 27 - DNA, 46 bp, Artificial Sequence
  Synthetic Construct
cagcggataacaatttcacacagggagctccaccgcggtggcggcc

SEQ ID NO: 28 - DNA, 23 bp, Artificial Sequence
  Synthetic Construct
cccagtcacgacgttgtaaaacg

SEQ ID NO: 29 - DNA, 19 bp, Artificial Sequence
  Synthetic Construct
ttttttttttttttttttv

SEQ ID NO: 30 - DNA, 35 bp, Artificial Sequence
  Synthetic Construct
ggtggcggccgcaggaatttttttttttttttttt

SEQ ID NO: 31 - DNA, 21 bp, Artificial Sequence
  Synthetic Construct
actgcaagggtccaatcacag

SEQ ID NO: 32 - DNA, 21 bp, Artificial Sequence
  Synthetic Construct
tcttgcctctggaatgctctg

SEQ ID NO: 33 - DNA, 19 bp, Artificial Sequence
  Synthetic Construct
gtgtggtggccttgtctgg

SEQ ID NO: 34 - DNA, 20 bp, Artificial Sequence
  Synthetic Construct
tggctgtcaccaaagagcag

SEQ ID NO: 35 - DNA, 20 bp, Artificial Sequence
  Synthetic Construct
ttcaggaccagaatgaggcg

SEQ ID NO: 36 - DNA, 19 bp, Artificial Sequence
  Synthetic Construct
aagtgcaagctggcagcaa

SEQ ID NO: 37 - DNA, 20 bp, Artificial Sequence
  Synthetic Construct
tgattgtttcccttccccag

SEQ ID NO: 38 - DNA, 21 bp, Artificial Sequence
  Synthetic Construct
cacctgtctcgaagtttgcct

SEQ ID NO: 39 - DNA, 20 bp, Artificial Sequence
  Synthetic Construct
ggagaccacgggaaattgct

SEQ ID NO: 40 - DNA, 20 bp, Artificial Sequence
  Synthetic Construct
cctagggctctcagctcgct

SEQ ID NO: 41 - DNA, 26 bp, Artificial Sequence
  Synthetic Construct
gatgtaccactaacagggatttaggg

SEQ ID NO: 42 - DNA, 24 bp, Artificial Sequence
  Synthetic Construct
gtggatccttaggtaaataggcca

SEQ ID NO: 43 - DNA, 20 bp, Artificial Sequence
  Synthetic Construct
gaacgtgccccttcctcata

SEQ ID NO: 44 - DNA, 22 bp, Artificial Sequence
  Synthetic Construct
tcaactctgcgtctcttgctgt

SEQ ID NO: 45 - DNA, 23 bp, Artificial Sequence
  Synthetic Construct
ccaataaggaaactctgaaccag

SEQ ID NO: 46 - DNA, 23 bp, Artificial Sequence
  Synthetic Construct
tttcaaacactgggaagtaacgg

SEQ ID NO: 47 - DNA, 22 bp, Artificial Sequence
  Synthetic Construct
ctagctttagcagtgaagaggg

SEQ ID NO: 48 - DNA, 23 bp, Artificial Sequence
  Synthetic Construct
tttgtcattgttggacagagtgg

SEQ ID NO: 49 - DNA, 21 bp, Artificial Sequence
  Synthetic Construct
ccactatgttgacgtggtcag

SEQ ID NO: 50 - DNA, 28 bp, Artificial Sequence
  Synthetic Construct
ttaacagtttttcactctatttaattgg